(19) United States
(12) Patent Application Publication
(10) Pub. No.: US 2007/0006348 A1
(43) Pub. Date: Jan. 4, 2007

(54) Environmental stress responsive promoter

(75) Inventors: Kazuo Shinozaki, Ibaraki (JP); Motoaki Seki, Ibaraki (JP); Tokihiko Nanjo, Ibaraki (JP)

Correspondence Address: KENYON & KENYON LLP, 1500 K STREET N.W., SUITE 700, WASHINGTON, DC, 20005, US

(73) Assignees: RIKEN,, Wako-shi (JP); TOYOTA JIDOSHA KABUSHIKI KAISHA, Toyota-shi (JP)

(21) Appl. No.: 09/988,739

(22) Filed: Nov. 20, 2001

(30) Foreign Application Priority Data
Nov. 22, 2000 (JP) 356652/2000
Oct. 5, 2001 (JP) 309984/2001

(51) Int. Cl. A01H 1/00; C07H 21/04; C12N 15/82; C12N 5/04

(52) U.S. Cl. 800/289; 435/419; 435/468; 536/23.6

(57) ABSTRACT

The present invention provides a stress responsive promoter. The environmental stress responsive promoter of the present invention comprises DNA of the following (a), (b) or (c): (a) DNA consisting of any nucleotide sequence selected from SEQ ID NOS: 1 to 18; (b) DNA consisting of a nucleotide sequence comprising a deletion, substitution or addition of one or more nucleotides relative to any nucleotide sequence selected from SEQ ID NOS: 1 to 18, and functioning as an environmental stress responsive promoter; and (c) DNA hybridizing under stringent conditions to DNA consisting of any nucleotide sequence selected from SEQ ID NOS: 1 to 18, and functioning as an environmental stress responsive promoter.

### FIELD OF THE INVENTION

The present invention relates to an environmental stress responsive promoter.

### BACKGROUND OF THE INVENTION

By means of gene sequencing projects, large quantities of genomic and cDNA sequences have been determined for a number of organisms, and in a plant model, Arabidopsis thaliana, the complete genomic sequences of two chromosomes have been determined (Lin, X. et al., (1999) Nature 402, 761-768; Mayer, K. et al., (1999) Nature 402, 769-777.)

The EST (expressed sequence tag) project has also contributed greatly to the discovery of expression genes (Hofte, H. et al., (1993) Plant J. 4, 1051-1061; Newman, T. et al., (1994) Plant Physiol. 106, 1241-1255; Cooke, R. et al., (1996) Plant J. 9, 101-124. Asamizu, E. et al., (2000) DNA Res. 7, 175-180.) For example, dbEST (the EST database of the National Center for Biotechnology Information (NCBI)) comprises partial cDNA sequences, in which more than a half (about 28,000 genes) of the total gene complement is represented (as estimated from the gene content of the completely sequenced Arabidopsis thaliana chromosome 2 (Lin, X. et al., (1999) Nature 402, 761-768.))

In recent years, microarray (DNA chip) technology has become a useful tool for analysis of genome-scale gene expression (Schena, M. et al., (1995) Science 270, 467-470; Eisen, M. B. and Brown, P. O. (1999) Methods Enzymol. 303, 179-205.) In this DNA chip-based technology, a cDNA sequence is arrayed on a glass slide at a density of more than 1,000 genes/cm². The thus arrayed cDNA sequence is hybridized simultaneously to a two-color fluorescently labeled cDNA probe pair of different cell or tissue type RNA samples, so as to allow direct and large-scale comparative analysis of gene expression. This technology was first demonstrated by analyzing 48 Arabidopsis genes in respect of differential expression in roots and shoots (Schena, M. et al., (1995) Science 270, 467-470.) Furthermore, microarrays were used to study 1,000 clones randomly selected from a human cDNA library for identification of novel genes responding to heat shock and protein kinase C activation (Schena M. et al., (1996) Proc. Natl. Acad. Sci. USA, 93, 10614-10619.)

In another study, expression profiles of inflammatory disease-related genes were analyzed under various induction conditions by this DNA chip-based method (Heller, R. A. et al., (1997) Proc. Natl. Acad. Sci. USA, 94. 2150-2155.) Moreover, the yeast genome of more than 6,000 coding sequences has also been analyzed in respect of dynamic expression by the use of microarrays (DeRisi, J. L. et al., (1997) Science 278, 680-686; Wodicka, L. et al., (1997) Nature Biotechnol. 15, 1359-1367.)

In plant science, however, only a few reports regarding microarray analysis have been published (Schena, M. et al., (1995) Science 270, 467-470; Ruan, Y. et al., (1998) Plant J. 15, 821-833; Aharoni. A. et al., (2000) Plant Cell 12, 647-661; Reymond, P. et al., (2000) Plant Cell 12, 707-719.)

Plant growth is greatly affected by environmental stresses such as drought, high salinity and low temperature. Among these stresses, drought or water deficiency is the most severe limiting factor for plant growth and crop production. Drought stress induces various biochemical and physiological responses in plants.

Plants acquire responsivity and adaptability to these stresses to survive under stress conditions. Recently, a number of genes responding to drought at a transcriptional level have been described (Bohnert, H. J. et al., (1995) Plant Cell 7, 1099-1111; Ingram, J., and Bartels, D. (1996) Plant Mol. Biol. 47, 377-403; Bray, E. A. (1997) Trends Plant Sci. 2, 48-54; Shinozaki. K., and Yamaguchi-Shinozaki, K. (1997) Plant Physiol. 115, 327-334; Shinozaki, K., and Yamaguchi-Shinozaki, K. (1999). Molecular responses to drought stress. Molecular responses to cold, drought, heat and salt stress in higher plants. Edited by Shinozaki, K. and Yamaguchi-Shinozaki, K. R. G. Landes Company; Shinozaki, K., and Yamaguchi-Shinozaki, K. (2000) Curr. Opin. Plant Biol. 3, 217-223.)

Stress-inducible genes have been used to improve stress tolerance of plants by gene transfer (Holmberg, N., and Bulow, L. (1998) Trends Plant Sci. 3, 61-66; Bajaj. S. et al., (1999) Mol. Breed. 5, 493-503.) It is important to analyze the functions of stress-inducible genes not only to understand the molecular mechanisms of stress tolerance and responses of higher plants, but also to improve the stress tolerance of crops by gene manipulation.

DRE/CRT (dehydration-responsive element/C-repeat sequence) has been identified as an important cis-acting element in drought-, high salt-, and cold stress-responsive gene expression in an ABA-independent manner (ABA refers to abscisic acid which is a kind of plant hormone and which acts as a signal transmission factor of seed dormancy and environmental stress) (Yamaguchi-Shinozaki, K., and Shinozaki, K. (1994) Plant Cell 6, 251-264; Thomashow, M. F. et al., (1999) Plant Mol. Biol. 50, 571-599; Shinozaki, K., and Yamaguchi-Shinozaki, K. (2000) Curr. Opin. Plant Biol. 3, 217-223.) Transcription factors (DREB/CBF) involved in DRE/CRT-responsive gene expression have been cloned (Stockinger. E. J. et al., (1997) Proc. Natl. Acad. Sci. USA 94, 1035-1040; Liu, Q. et al., (1998) Plant Cell 10, 1391-1406; Shinwari, Z. K. et al., (1998) Biochem. Biophys. Res. Commun. 250, 161-170; Gilmour, S. J. et al., (1998) Plant J. 16, 433-443.) DREB1/CBFs are considered to function in cold-responsive gene expression, whereas DREB2s are involved in drought-responsive gene expression. Strong tolerance to freezing stress was observed in transgenic Arabidopsis plants that overexpress CBF1 (DREB1B) cDNA under the control of a cauliflower mosaic virus (CaMV) 35S promoter (Jaglo-Ottosen, K. R. et al., (1998) Science 280, 104-106.)

The present inventors have reported that overexpression of the DREB1A (CBF3) cDNA molecules in transgenic plants under the control of a CaMV 35S promoter or a stress-inducible rd29A promoter gave rise to strong constitutive expression of the stress-inducible DREB1A target genes and increased tolerance to freezing, drought and salt stresses (Liu, Q. et al., (1998) Plant Cell 10, 1391-1406; Kasuga, M. et al., (1999) Nature Biotechnol. 17, 287-291.) Furthermore, the present inventors have already identified six DREB1A target genes such as rd29A/lti78/cor78, kin1, kin2/cor6.6, cor15a, rd17/cor47 and erd10 (Kasuga, M. et al., (1999) Nature Biotechnol. 17, 287-291.) However, it is not well clarified how overexpression of the DREB1A cDNA molecules in transgenic plants increases stress tolerance to freezing, drought and salt. To study the molecular mechanisms of drought and freezing tolerance, it is important to identify and analyze as many genes controlled by DREB1A as possible.

### SUMMARY OF THE INVENTION

The present invention is directed to providing an environmental stress responsive promoter.

Through intensive studies directed toward the above object, the present inventors have succeeded in identifying a novel DREB1A target gene and isolating a promoter region thereof by applying cDNA microarray analysis, thereby completing the present invention.

That is to say, the present invention is an environmental stress responsive promoter comprising DNA of the following (a), (b) or (c):

(a) DNA consisting of any nucleotide sequence selected from SEQ ID NOS: 1 to 18;

(b) DNA consisting of a nucleotide sequence comprising a deletion, substitution or addition of one or more nucleotides relative to any nucleotide sequence selected from SEQ ID NOS: 1 to 18, and functioning as an environmental stress responsive promoter; and

(c) DNA hybridizing under stringent conditions to DNA consisting of any nucleotide sequence selected from SEQ ID NOS: 1 to 18, and functioning as an environmental stress responsive promoter.

The environmental stress is at least one selected from the group consisting of cold stress, drought stress, salt stress and high photo stress.

Moreover, the present invention is an expression vector comprising the above promoter, or the expression vector further comprising a desired gene.

Furthermore, the present invention is a transformant comprising the above expression vector.

Still further, the present invention is a transgenic plant (e.g. a plant body, plant organ, plant tissue or plant culture cell) comprising the above expression vector.

Moreover, the present invention is a method for producing a stress-resistant plant, which comprises culturing or cultivating the above transgenic plant.

### BRIEF DESCRIPTION OF THE DRAWINGS

FIG. 1 is a photograph showing results of cDNA microarray analysis of gene expression under cold stress.

FIG. 2 is a figure showing strategy for the identification of drought- or cold-inducible genes and DREB1A target genes.

FIG. 3 is a photograph showing a comparison of cDNA microarray and Northern Blot analysis for new DREB1A target genes and a DREB1A gene.

FIG. 4 is a figure showing results of classification of the identified drought- or cold-inducible genes into four groups on the basis of RNA gel blot and microarray analyses.

FIG. 5 shows the relation between cold treatment period and expression rate regarding FL3-5A3.

FIG. 6 shows the relation between dehydration treatment period and expression rate regarding FL3-5A3.

FIG. 7 shows the relation between high salt treatment period and expression rate regarding FL3-5A3.

FIG. 8 shows the relation between cold treatment period and expression rate regarding FL5-2H15.

FIG. 9 shows the relation between dehydration treatment period and expression rate regarding FL5-2H15.

FIG. 10 shows the relation between high salt treatment period and expression rate regarding FL5-2H15.

FIG. 11 shows the relation between dehydration treatment period and expression rate regarding FL5-3M24.

FIG. 12 shows the relation between high salt treatment period and expression rate regarding FL5-3M24.

FIG. 13 shows the relation between cold treatment period and expression rate regarding FL5-90.

FIG. 14 shows the relation between cold treatment period and expression rate regarding FL5-2I22.

FIG. 15 shows the relation between dehydration treatment period and expression rate regarding FL5-2I22.

FIG. 16 shows the relation between high salt treatment period and expression rate regarding FL5-2I22.

FIG. 17 shows the relation between dehydration treatment period and expression rate regarding FL6-55.

FIG. 18 shows the relation between high salt treatment period and expression rate regarding FL6-55.

FIG. 19 shows the relation between dehydration treatment period and expression rate regarding FL1-159.

FIG. 20 shows the relation between dehydration treatment period and expression rate regarding FL5-2D23.

FIG. 21 shows the relation between high salt treatment period and expression rate regarding FL5-2D23.

FIG. 22 shows the relation between dehydration treatment period and expression rate regarding FL05-08-P24.

FIG. 23 shows the relation between dehydration treatment period and expression rate regarding FL05-09-G08.

FIG. 24 shows the relation between dehydration treatment period and expression rate regarding FL05-09-P10.

FIG. 25 shows the relation between ABA treatment period and expression rate regarding FL05-09-P10.

FIG. 26 shows the relation between high salt treatment period and expression rate regarding FL05-10-N02.

FIG. 27 shows the relation between dehydration treatment period and expression rate regarding FL05-18-I12.

FIG. 28 shows the relation between high salt treatment period and expression rate regarding FL05-18-I12.

FIG. 29 shows the relation between ABA treatment period and expression rate regarding FL05-18-I12.

FIG. 30 shows the relation between dehydration treatment period and expression rate regarding FL05-21-F13.

FIG. 31 shows the relation between cold treatment period and expression rate regarding FL05-21-F13.

FIG. 32 shows the relation between dehydration treatment period and expression rate regarding FL06-10-C16.

FIG. 33 shows the relation between high salt treatment period and expression rate regarding FL06-10-C16.

FIG. 34 shows the relation between ABA treatment period and expression rate regarding FL06-10-C16.

FIG. 35 shows the relation between dehydration treatment period and expression rate regarding FL06-15-P15.

FIG. 36 shows the relation between high salt treatment period and expression rate regarding FL06-15-P15.

FIG. 37 shows the relation between ABA treatment period and expression rate regarding FL06-15-P15.

FIG. 38 shows the relation between dehydration treatment period and expression rate regarding FL08-10-E21.

FIG. 39 shows the relation between high salt treatment period and expression rate regarding FL09-11-P10.

### DETAILED DESCRIPTION OF THE INVENTION

Hereinafter, the present invention is described in detail.

Applying the biotinylated CAP trapper method (Carninci. P. et al., (1996) Genomics, 37, 327-336.), the present inventors constructed full-length cDNA libraries from Arabidopsis plants under different conditions, such as drought-treated and cold-treated plants, etc. (Seki. M. et al., (1998) Plant J. 15, 707-720.) Using about 1,300 full-length cDNA molecules and about 7,000 full-length cDNA molecules which both contained stress-inducible genes, the present inventors prepared an Arabidopsis full-length cDNA microarray for each case. In addition to these drought- and cold-inducible full-length cDNA molecules, the present inventors also prepared another cDNA microarray, using a DREB1A target gene, a transcriptional regulator for controlling expression of a stress-responsive gene. Thereafter, expression patterns of genes under drought- and cold-stresses were monitored to exhaustively analyze stress-responsive genes. As a result, novel environmental stress responsive genes, that is, 44 drought-inducible genes and 19 cold-inducible genes were isolated from a full-length cDNA microarray containing about 1,300 full-length cDNA molecules. 30 out of the 44 drought-inducible genes and 10 out of the 19 cold-inducible genes, were novel stress-inducible genes. Moreover, it was found that 12 stress-inducible genes were DREB1A target genes, and 6 of these 12 were novel genes. Furthermore, as a result of this analysis, 301 drought-inducible genes, 54 cold-inducible genes and 211 high salt-inducible genes were isolated from a cDNA microarray containing about 7,000 full-length cDNA molecules.

Thereafter, promoter regions were successfully isolated from these environmental stress responsive genes.

As stated above, a full-length cDNA microarray is a useful tool for analysis of the expression manner of Arabidopsis thaliana drought- and cold-stress inducible genes, and analysis of the target gene of a stress-related transcriptional regulator.

1. Isolation of Promoter

The promoter of the present invention is a cis-element existing upstream of a gene encoding a stress-responsive protein expressed by environmental stresses such as cold-, drought- and high salt-stresses, and the cis-element has a function of binding to a transcriptional factor to activate transcription of a gene existing downstream. Examples of such cis-elements include a drought stress responsive element (DRE; dehydration-responsive element), an abscisic acid responsive element (ABRE), and a cold stress responsive element, etc. Examples of genes encoding proteins binding to these elements include a DRE binding protein 1A gene (referred to also as a DREB1A gene), a DRE binding protein 1C gene (referred to also as a DREB1C gene), a DRE binding protein 2A gene (referred to also as a DREB2A gene) and a DRE binding protein 2B gene (referred to also as a DREB2B gene), etc.

For isolation of the promoter of the present invention, first, stress responsive genes are isolated using a microarray. For preparation of a microarray, there can be used about 1,300 cDNA molecules in total, being genes isolated from Arabidopsis full-length cDNA libraries, RD (responsive to dehydration) genes, ERD (early responsive to dehydration) genes, kin1 genes, kin2 genes, cor15a genes, α-tubulin genes as an internal standard, and as negative controls, epsilon subunit (nAChRE) genes of a mouse acetylcholine nicotinate receptor and homologous genes of a mouse glucocorticoid receptor.

As a microarray used to isolate the promoter of the present invention, there can be used about 7,000 cDNA molecules in total, being genes isolated from Arabidopsis full-length cDNA libraries, RD (responsive to dehydration) genes, ERD (early responsive to dehydration) genes, and PCR amplification fragments as an internal standard obtained from A control template DNA fragments (TX803, Takara Shuzo), and as negative controls, epsilon subunit (nAChRE) genes of a mouse acetylcholine nicotinate receptor and homologous genes of a mouse glucocorticoid receptor.

A plasmid DNA extracted with a plasmid preparation device (Kurabo) is sequenced by sequence analysis, using a DNA sequencer (ABI PRISM 3700, PE Applied Biosystems, CA, USA). Based on the GenBank/EMBL database, homology detection of the obtained sequence is carried out with the BLAST program.

After poly A selection, reverse transcription is carried out to synthesize double-stranded DNA molecules, and a cDNA molecule is inserted into a vector.

The cDNA molecule inserted into a vector for preparation of cDNA libraries is amplified by PCR, using primers complementary to sequences of vectors on both sides of the cDNA molecule. Examples of such vectors include λZAPII and λPS, etc.

A microarray can be prepared according to ordinary methods and so the method is not particularly limited. For example, using a gene tip microarray stamp machine, GTMASS SYSTEM (Nippon Laser & Electronics Lab.), the above obtained PCR product is loaded from a microtiter plate and spotted on a micro slide glass at regular intervals. Then, to prevent expression of non-specific signals, the slide is immersed into a blocking solution.

Examples of plant materials include plant strains obtained by destroying specific genes as well as wild type plants, and there can be used a transgenic plant, into which cDNA of DREB1A is introduced. Examples of plant varieties include Arabidopsis thaliana, tobacco and rice, etc., and Arabidopsis thaliana is preferable.

Drought- and cold-stress treatments can be carried out according to a known method (Yamaguchi-Shinozaki, K., and Shinozaki, K. (1994) Plant Cell 6, 251-264.)

After performing the stress treatments, plant bodies (wild type plants and DREB1A overexpression transformants) are sampled, and are subjected to cryopreservation with liquid nitrogen. The wild type plants and the DREB1A overexpression transformants are used for an experiment to identify DREB1A target genes. According to a known method or using a kit, mRNA is isolated from plant bodies and purified.

In the presence of Cy3 dUTP or Cy5 dUTP for labeling (Amersham Pharmacia), each of the mRNA samples is subjected to reverse transcription and then used for hybridization.

After hybridization, the microarray is scanned with a scanning laser microscope. As a program for analyzing data of a microarray, Imagene Ver 2.0 (BioDiscovery) and QuantArray (GSI Lumonics), etc. can be used.

After scanning, genes of interest are isolated by preparation of a plasmid comprising the gene.

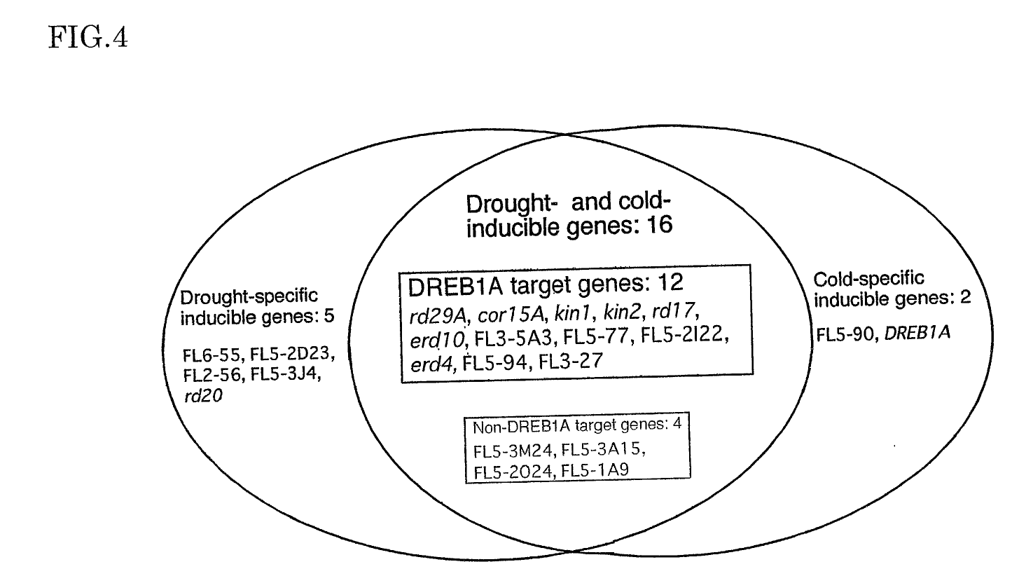

Determination of a promoter region is carried out by analysis of the nucleotide sequences of the above isolated genes, followed by the use of a gene analysis program based on the genomic information of database (GenBank/EMBL, ABRC). The isolated genes can be classified into ones having both drought- and cold-stress inductivity, ones specific for drought stress inductivity, and ones specific for cold stress inductivity (FIG. 4). According to a gene analysis program, 18 types of genes (FL3-5A3, FL5-2H15, FL5-3M24, FL5-90, FL5-2I22, FL6-55, FL1-159, FL5-2D23, FL05-08P-24, FL05-09-G08, FL05-09-P10, FL05-10-NO₂, FL05-18-I12, FL05-21-F13, FL06-10-C16, FL06-15-P15, FL08-10-E21 and FL09-11-P10) are identified from the above genes. The promoter regions of these genes are shown in SEQ ID NOS: 1 to 18, respectively.

As long as the promoter of the present invention acts as an environmental stress responsive promoter, it may be a promoter having a nucleotide sequence comprising a deletion, substitution or addition of one or more nucleotides, preferably one or several nucleotides (e.g. 1 to 10 nucleotides, preferably 1 to 5 nucleotides) relative to any nucleotide sequence selected from SEQ ID NOS: 1 to 18. Furthermore, the promoter of the present invention also includes DNA hybridizing under stringent conditions to the DNA comprising any nucleotide sequence selected from SEQ ID NOS: 1 to 18 and further acting as an environmental stress responsive promoter.

Once the nucleotide sequence of the promoter of the present invention is determined, then the promoter itself can be obtained by chemical synthesis, PCR using a cloned probe as a template, or hybridization, using as a probe, DNA fragments having the nucleotide sequence. Furthermore, a mutant of the present promoter, which has functions equivalent to those of a non-mutated promoter, can also be synthesized by a site-directed mutagenesis, etc.

To introduce a mutation into a promoter sequence, the known methods such as the Kunkel method and Gapped duplex method, or an equivalent method, can be applied. Introduction of a mutation can be carried out, for example, using a kit for introducing mutant (e.g. Mutant-K (Takara) and Mutant-G (Takara)) by a site-directed mutagenesis or using the LA PCR in vitro Mutagenesis series kit (Takara).

The term “function as an environmental stress responsive promoter” is used herein to mean a function of binding RNA polymerase to a promoter to allow initiation of transcription, when the promoter is exposed to a specific environmental stress condition.

The term “environmental stress” is used generally to mean an abiotic stress such as drought stress, cold stress, high salt stress, high photo stress, etc. The term “drought” is used herein to mean a water deficient state, while the term “cold” is used herein to mean a state of being exposed to a lower temperature than the optimum living temperature of each organism variety (e.g., in the case of Arabidopsis thaliana, it is continuously exposed at −20 to +21° C. for 1 hour to several weeks). The term “high salt” is used herein to mean a state after continuous treatment with NaCl having a concentration of 50 mM to 600 mM for 0.5 hours to several weeks. The term “high photo stress” is used herein to mean a state wherein a strong light greater than its photosynthetic ability is applied to a plant, and an example is application of a strong light of more than 5,000 to 10,000 1×. With regard to these environmental stresses, one kind of stress may be loaded, or several kinds of stresses may be loaded.

The plant promoter of the present invention includes a promoter comprising an addition of a nucleotide sequence which increases translation efficiency at the 3′-terminus of any nucleotide sequence of SEQ ID NOS: 1 to 18, or a promoter retaining a promoter activity thereof while deleting a 5′-terminus thereof.

Furthermore, the promoter of the present invention includes DNA which hybridizes under stringent conditions to DNA consisting of any nucleotide sequence selected from SEQ ID NOS: 1 to 18, and functions as an environmental stress responsive promoter. The term “stringent conditions” used herein means sodium concentration of 25 to 500 mM, preferably 25 to 300 mM, and temperature of 42° C. to 68° C., preferably 42° C. to 65° C. More specifically, such conditions are 5×SSC (83 mM NaCl, 83 mM sodium citrate) and temperature of 42° C.

2. Construction of Expression Vector

The expression vector of the present invention can be obtained by ligation (insertion) of the promoter of the present invention to an appropriate vector. As long as a vector into which the promoter of the present invention is inserted is capable of replicating in a host, it is not particularly limited, and examples of vectors include a plasmid, a shattle vector and a helper plasmid, etc.

Examples of plasmid DNA include a plasmid derived from Escherichia coli (e.g. pBR322, pBR325, pUC118, pUC119, pUC18, pUC19 and pBluescript, etc.), a plasmid derived from Bacillus subtilis (e.g. pUB110 and pTP5, etc.), and a plasmid derived from yeast (e.g. YEp13 and YCp50, etc.), and examples of phage DNA include λphage (e.g. Charon4A, Charon21A, EMBL3, EMBL4, λgt10, λgt11 and λZAP, etc.) Further, animal virus vectors such as a retrovirus and a vaccinia virus, and insect virus vectors such as a baculovirus can also be used.

To insert the promoter of the present invention into a vector, there is applied a method in which, first, the purified DNA is cleaved with suitable restriction enzymes, and, next, the obtained DNA fragment is inserted into the restriction enzyme site of a suitable vector DNA or a multi-cloning site so as to ligate to the vector.

In the present invention, in order to express a desired gene, the desired gene can be further inserted into the above expression vector. The technique involving insertion of a desired gene is the same as the method involving insertion of a promoter into a vector. A desired gene is not particularly limited, and examples of the gene include genes shown in Table 2 and the known genes other than those, etc.

In a case where the promoter of the present invention is used with a reporter gene, e.g. a GUS gene widely used in plant science, ligated to a 3′-terminus thereof, the strength of the promoter can easily be determined by examining a GUS activity. As a reporter gene, not only a GUS gene but also luciferase and a green fluorescent protein can be used.

Thus, various types of vectors can be used in the present invention. Further, there can be prepared a product by connecting a desired gene of interest to the promoter of the present invention in a sense or antisense direction, and thereafter such product can be inserted into a vector called a binary vector, such as pBI101 (Clonetech).

3. Preparation of Transformant

The transformant of the present invention can be obtained by introduction of the expression vector of the present invention into a host. A host herein is not particularly limited, as long as it can express a promoter or gene of interest, a plant being preferable. Where a host is a plant, a transformant plant (a transgenic plant) can be obtained as follows.

A plant to be transformed in the present invention means any of an entire plant, a plant organ (e.g. a leaf, a petal, a stem, a root, a seed, etc.), a plant tissue (e.g., an epidermis, a phloem, a parenchyma, a xylem, a vascular bundle, etc.) and a plant culture cell. Examples of plants used for transformation include plants belonging to Brassicaceae, Gramineae, Solanaceae and Leguminosae, etc. (see below), but are not limited thereto.

Brassicaceae: Arabidopsis thaliana

Gramineae: Nicotiana tabacum

Solanaceae: Zea mays, Oryza sativa

Leguminosae: Glycine max

The above recombinant vector can be introduced into a plant by ordinary transformation methods such as electroporation, Agrobacterium method, particle gun method, PEG, etc.

For example, where electroporation is applied, using an electroporation device equipped with a pulse controller, a vector is processed under conditions of a voltage of 500 to 1,600V, 25 to 1,000 μF and 20 to 30 msec, and a gene is introduced into a host.

Where a particle gun method is applied, a plant body, plant organ or plant tissue may be used as is, after preparation of a section, or a protoplast may be prepared. The thus prepared sample can be processed with a gene-introduction device (e.g. PDS-1000/He, Bio-Rad, etc.) Conditions for processing depend on a plant or sample, but generally, a pressure of about 1,000 to 1,800 psi and a distance of 5 to 6 cm are applied as processing conditions.

A gene of interest can be introduced into a plant by using a plant virus as a vector. Examples of available plant viruses include a cauliflower mosaic virus. That is, first, a virus genome is inserted into a vector derived from Escherichia coli to prepare a recombinant, and then the gene of interest is inserted into the virus genome. The thus modified virus genome is cut from the recombinant with restriction enzymes, and inoculated into a plant host, so that a gene of interest can be introduced therein.

When bacteria belonging to Agrobacterium are transfected to a plant, the bacteria introduce a portion of plasmid DNA thereof into a plant genome. In a method involving the use of Ti plasmid of Agrobacterium, using such a character, a gene of interest is introduced into a plant host. Among bacteria belonging to Agrobacterium, Agrobacterium tumefaciens transfects to a plant and forms therein a tumor called a crown gall, whereas Agrobacterium rhizogenes transfects to a plant to generate hairy roots. These phenomena originate from a cause whereby a region called a T-DNA region (a transferred DNA region) located on a plasmid called a Ti plasmid or Ri plasmid existing in each bacterium is transferred into a plant and incorporated into a plant genome at a time of transfection.

By inserting DNA to be incorporated into a plant genome, into the T-DNA region of a Ti or Ri plasmid, DNA of interest can be incorporated into a plant genome, when Agrobacterium bacteria are transfected to a plant host.

Tumoral tissues, shoots and hairy roots obtained as a result of transformation can directly be used for cell culture, tissue culture or organ culture, and according to the previously known plant tissue culture method, a plant body can be regenerated by administration of a plant hormone (e.g. auxin, cytokinin, gibberellin, abscisic acid, ethylene, brassinoride, etc.) in a suitable concentration.

The vector of the present invention cannot only be introduced into the above-stated plant hosts, but can also be introduced into bacteria belonging to Escherichia such as Escherichia coli, Bacillus such as Bacillus subtilis and Pseudomonas such as Pseudomonas putida; yeast such as Saccharomyces cerevisiae and Schizosaccharomyces pombe; animal cells such as COS cell and CHO cell; and insect cells such as Sf9 cell, so that a transformant can be obtained. Where a bacterium such as Escherichia coli or yeast is used as a host, it is preferable that the recombinant vector of the present invention is capable of self-replicating in the bacterium and, at the same time, is also comprised of the promoter of the present invention, a ribosome binding sequence, a gene of interest and a transcription termination sequence. Furthermore, it may also comprise a gene for controlling a promoter.

A method for introduction of a recombinant vector into bacteria is not particularly limited, as long as it is a method for introduction of DNA into bacteria. For example, a method involving the use of calcium ions and an electroporation method can be applied.

Where yeast is used as a host, Saccharomyces cerevisiae and Schizosaccharomyces pombe, etc. can be used. A method for introduction of a recombinant vector into yeast is not particularly limited, as long as it is a method for introduction of DNA into yeast, and examples of such methods include electroporation, spheroplast method, lithium phosphate method, etc.

Where an animal cell is used as a host, a monkey COS-7 cell, Vero, a Chinese hamster ovary cell (a CHO cell), a mouse L cell, etc. are used. Examples of methods for introduction of a recombinant vector into an animal cell include electroporation, calcium phosphate method, lipofection method, etc.

Where an insect cell is a host, a Sf9 cell and the like can be used. Examples of methods for introduction of a recombinant vector into an insect cell include calcium phosphate method, lipofection method, electroporation, etc.

Confirmation regarding whether a gene is incorporated into a host or not can be carried out by methods such as PCR, Southern hybridization, Northern hybridization, etc. For example, DNA is prepared from a transformant, and DNA specific primers are designed for use with PCR. PCR is carried out under the same conditions as used for preparation of the above plasmid. Thereafter, the obtained amplified product is subjected to agarose gel electrophoresis, polyacrylamide gel electrophoresis or capillary electrophoresis, so that the product is stained with ethidium bromide or a SYBR Green solution, etc. Then, it is confirmed that the amplified product was transformed, by detecting the product as a single band. Or, the amplified product can also be detected by PCR, using primers stained with fluorescent dye or the like beforehand. Furthermore, there may also be adopted a method in which the amplified product is bound to a solid phase such as a microplate and confirmed by fluorescent or enzymic reaction, etc.

4. Production of Plant

In the present invention, a transformed plant body can be regenerated from the above transformed plant cell and the like. An adaptable regeneration method is one in which transformed cells are transferred to and cultured in media with different types of hormones and concentrations to promote nucellular embryony, thereby obtaining an entire plant body. Examples of applicable media include an LS medium and an MS medium, etc.

The “method for producing a plant body” of the present invention comprises processes of, introducing a plant expression vector, into which the above plant promoter is inserted, into a host cell to obtain a transformed plant cell; regenerating a transformed plant body from the transformed plant cell; obtaining plant seeds from the resulting transformed plant body; and producing a plant body from the plant seed.

To obtain a plant seed from a transformed plant body, for example, a transformed plant body is collected from a rooting medium and transferred to a pot with water-containing soil. Then, the transformed plant body is grown at constant temperature to form flowers, thereby finally obtaining seeds. To produce a plant body from a seed, for example, after a seed formed in a transformed plant body has matured, the seed is isolated and implanted in water-containing soil, followed by growing at constant temperature under illumination. The thus bred plant becomes an environmental stress-resistant plant corresponding to the stress responsivity of a promoter introduced.

### EXAMPLES

The present invention is further described in the following examples. The examples are not intended to limit the scope of the invention.

### Example 1

### Isolation of Promoter

1. Materials and methods

(1) Arabidopsis cDNA Clone

For preparation of a microarray, there were used about 1,300 cDNA molecules in total, which are genes isolated from Arabidopsis full-length cDNA libraries, RD (responsive to dehydration) genes, ERD (early responsive to dehydration) genes, kin1 genes, kin2 genes, cor15a genes, and α-tubulin genes as an internal standard, and as negative controls, epsilon subunit (nAChRE) genes of a mouse acetylcholine nicotinate receptor and homologous genes of a mouse glucocorticoid receptor.

Positive control: drought-inducible genes (responsive-to-dehydration genes: rd, and early responsive-to-dehydration genes: erd)

Internal standard: α-tubulin genes

Negative control: for analysis of non-specific hybridization, acetylcholine nicotinate receptor E subunit (nAChRE) genes and mouse glucocorticoid receptor homolog genes, which do not substantially have homology with any given sequence in Arabidopsis database.

Furthermore, for preparation of a microarray, there were used about 7,000 cDNA molecules in total, which are genes isolated from Arabidopsis full-length cDNA libraries, RD (responsive to dehydration) genes, ERD (early responsive to dehydration) genes, and PCR amplification fragments (hereinafter referred to as PCR fragments) as an internal standard obtained from λ control template DNA fragments (TX803, Takara Shuzo), and as negative controls, epsilon subunit (nAChRE) genes of a mouse acetylcholine nicotinate receptor and homologous genes of a mouse glucocorticoid receptor.

Positive control: drought-inducible genes (responsive-to-dehydration genes: rd, and early responsive-to-dehydration genes: erd)

Internal standard: PCR fragments

Negative control: for analysis of non-specific hybridization, acetylcholine nicotinate receptor ε subunit (nAChRE) genes and mouse glucocorticoid receptor homolog genes, which do not substantially have homology with any given sequence in the Arabidopsis database.

(2) Arabidopsis Full-Length cDNA Microarray

According to the biotinylated CAP trapper method, the present inventor has constructed a full-length cDNA library from Arabidopsis plant bodies under different conditions (e.g. dehydration treatment, cold treatment and untreatment in various growth stages from budding to mature seeds). The present inventor has independently isolated each of about 1,300 and about 7,000 Arabidopsis full-length cDNA molecules from a full-length cDNA library. According to the known method (Eisen and Brown, 1999), cDNA fragments amplified by PCR were arranged on a slide glass. The present inventor has prepared both a full-length cDNA microarray containing about 1,300 Arabidopsis full-length cDNA molecules and another full-length cDNA microarray containing about 7,000 Arabidopsis full-length cDNA molecules, which comprise the genes stated below.

(3) Isolation of Drought-, Cold- and High Salt-Inducible Genes Using cDNA Microarray

In this example, using a full-length cDNA microarray containing about 1,300 Arabidopsis full-length cDNA molecules, drought- and cold-inducible genes were isolated. Further, using a full-length cDNA microarray containing about 7,000 Arabidopsis full-length cDNA molecules, drought-, cold- and high salt-inducible genes were isolated.

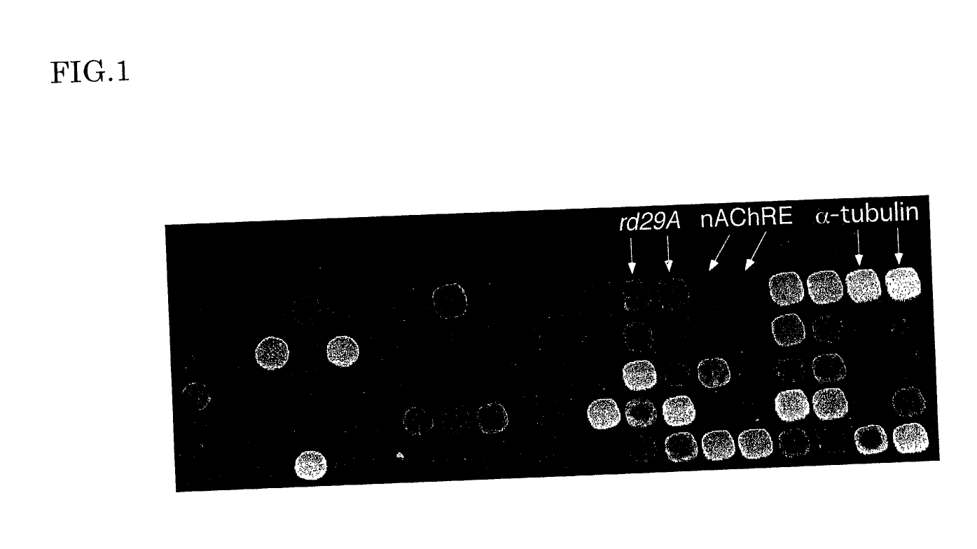

Both Cy3 and Cy5 fluorescent labeled probes of drought treated, cold treated and untreated plants were mixed, and the obtained mixture was hybridized to a full-length cDNA microarray containing about 1,300 Arabidopsis full-length cDNA molecules. FIG. 1 shows an image of the cDNA microarray. By the double labeling of a pair of cDNA probes, wherein one mRNA sample is labeled with Cy3-dUTP and the other mRNA sample is labeled with Cy5-dUTP, simultaneous hybridization to DNA elements on a microarray becomes possible and direct assay of gene expression level under two different conditions (that is, stressed and unstressed) is facilitated. The Cy3 and Cy5 emissions of each DNA element on the hybridized microarray was scanned using two different laser channels. Thereafter, the intensity rate of the two fluorescent signals of each DNA element was determined as a relative value, and then the change of differential expression of genes was determined, which was shown as a cDNA spot on a microarray. In this example, there was used, as an internal control gene, an α-tubulin gene, the expression level of which remains almost constant under two different experimental conditions.

In the case of a full-length cDNA microarray containing about 7,000 Arabidopsis full-length cDNA molecules, Cy3 and Cy5 fluorescent labeled probes of each of a dehydration treated plant, a cold treated plant, a high salt-inducible gene and an unstressed plant were mixed and the obtained mixture hybridized. A PCR fragment was used as an internal control gene in this cDNA microarray.

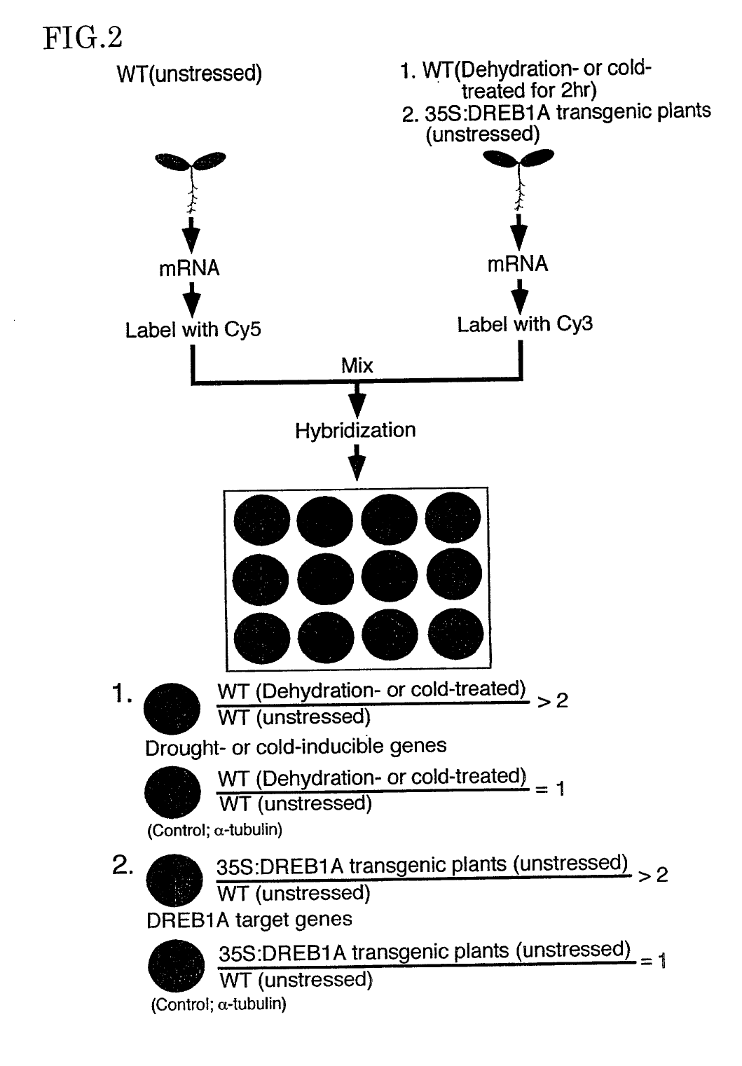

FIG. 2 shows the identification process of drought- or cold-inducible genes in a full-length cDNA microarray containing about 1,300 Arabidopsis full-length cDNA molecules. Furthermore, in the case of a full-length cDNA microarray containing about 7,000 Arabidopsis full-length cDNA molecules also, the identification of drought-, cold- or high salt-inducible genes was performed according to the same process as shown in FIG. 2.

1) Both mRNA molecules derived from a dehydration- or cold-treated plant and mRNA molecules derived from an unstressed wild type plant were used to prepare both Cy3- and Cy5-labeled cDNA probes. These cDNA probes were mixed, and then hybridized to a cDNA microarray. In this example, there was used, as an internal control gene, an α-tubulin gene, the expression level of which remains almost constant under two different experimental conditions. A gene having more than double the expression level (drought/unstressed or cold/unstressed) of an α-tubulin gene was defined as a drought- or cold-inducible gene (FIG. 2).

2) Both mRNA molecules derived from a 35S:DREB1A transgenic plant and mRNA molecules derived from an unstressed wild type plant were used to prepare both Cy3- and Cy5-labeled cDNA probes. These cDNA probes were mixed, and then hybridized to a cDNA microarray. In this example, there was used, as an internal control gene, an α-tubulin gene, the expression level of which remains almost constant under two different experimental conditions. A gene having an expression level in a 35S:DREB1A transgenic plant of more than double its expression level in an unstressed wild type plant was defined as a DREB1A target gene (FIG. 2).

Both mRNA molecules derived from a dehydration- or cold-treated plant and mRNA molecules derived from an unstressed wild type plant were used to prepare both Cy3- and Cy5-labeled cDNA probes. These cDNA probes were mixed, and then hybridized to a cDNA microarray. To evaluate reproducibility of microarray analysis, the same experiment was repeated five times. When the same mRNA sample was hybridized to various types of microarrays, a good correlation was observed. A gene having more than double the expression level (drought/unstressed or cold/unstressed) of an α-tubulin gene was defined as a drought- or cold-inducible gene (FIG. 2).

(4) Analysis of Sequence

To perform homology detection of gene sequences, a plasmid DNA extracted with a plasmid preparation device (NA 100, Kurabo) was used for sequence analysis. A DNA sequence was determined by dye terminator cycle sequencing method, using a DNA sequencer (ABI PRISM 3700, PE Applied Biosystems, CA, USA). Based on the GenBank/EMBL database, homology detection of sequences was carried out using the BLAST program.

(5) Amplification of cDNA

As a vector for preparation of cDNA libraries, λZAPII (Carninci et al., 1996) was used. The cDNA inserted into a vector for libraries was amplified by PCR, using primers complementary to vector sequences located on both sides of the cDNA.

The sequences of primers are as follows:

| | |
|---|---|
| FL forward 1224: | |
| 5′-CGCCAGGGTTTTCCCAGTCACGA | (SEQ ID NO: 19) |
| FL reverse 1233: | |
| 5′-AGCGGATAACAATTTCACACAGGA | (SEQ ID NO: 20) |

As a template, a plasmid (1 to 2 ng) was added to 100 μl of PCR mixture (0.25 mM dNTP, 0.2 μM PCR primers, 1×Ex Taq buffer, 1.25 U Ex Taq polymerase (Takara Shuzo)). PCR was performed under the following conditions: initial reaction at 94° C. for 3 minutes, 35 cycles of 95° C. for 1 minute, 60° C. for 30 seconds and 72° C. for 3 minutes, and the final reaction at 72° C. for 3 minutes. After precipitation of a PCR product with ethanol, the precipitate was dissolved into 25 μl of 3×SSC. 0.7% agarose gel electrophoresis was performed to confirm the quality of the obtained DNA and amplification efficiency of PCR.

(6) Production of cDNA Microarray

Using a gene tip microarray stamp machine, GTMASS SYSTEM (Nippon Laser & Electronics Lab.), 0.5 μl of PCR product (100 to 500 ng/ml) was loaded from a 384-well microtiter plate, and 5 nl each of the product was spotted on 6 micro slide glasses (S7444, Matsunami) coated with poly-L-lysine at a space of 280 μm. To spot an equivalent amount of DNA, after printing, slides were wetted in a beaker containing hot distilled water and then dried at 100° C. for 3 seconds. Thereafter, the slides were placed on a slide rack, and the rack was placed into a glass chamber. Then, a blocking solution (containing 15 ml of 1M sodium borate salt (pH8.0), 5.5 g of succinic anhydrous compound (Wako), and 335 ml of 1-methyl-2-pyrrolidone (Wako)) was poured into the glass chamber. After shaking the glass chamber containing the slides rack up and down 5 times, it was further shaken gently for 15 minutes. Thereafter, the slide rack was transferred to a glass chamber containing boiling water and shaken 5 times, followed by being left at rest for 2 minutes. Then, the slide rack was transferred to a glass chamber containing 95% ethanol and shaken 5 times, followed by centrifugation (800 rpm) for 30 minutes.

(7) Plant Materials and Isolation of RNA

As plant materials, there were used both a wild type Arabidopsis thaliana plant body which was seeded on an agar medium and grown for 3 weeks (Yamaguchi-Shinozaki and Shinozaki, 1994), and an Arabidopsis thaliana (Colombian species) plant body, into which DREB1A cDNA (Kasuga et al., 1999) connected to 35S promoter of a cauliflower mosaic virus was introduced. Drought- and cold-stress treatments were performed by the method of Yamaguchi-Shinozaki and Shinozaki (1994). That is to say, a plant body pulled out of an agar medium was placed on a filter and then dehydration treatment was carried out under conditions of 22° C. and 60% relative humidity. Cold treatment was carried out by transferring the plant grown at 22° C. into conditions of 4° C. High salt-stress treatment was carried out by water-culturing in a solution containing 250 mM NaCl.

After wild type plant bodies were exposed to stress-treatment for 2 or 10 hours, they were subjected to sampling, and then subjected to cryopreservation with liquid nitrogen. Both wild type and DREB1A overexpression type transformants, which were cultured in an agar medium without kanamycin, were used for an experiment for identification of a DREB1A target gene. Stress treatment was not performed for DREB1A overexpression type transformants. The total RNA was isolated from plant bodies using ISOGEN (Nippon gene, Tokyo, Japan), and then mRNA was isolated and purified using an Oligotex-dT30 mRNA purification kit (Takara, Tokyo, Japan).

(8) Fluorescent Labeling of Probe

In the presence of Cy3 dUTP or Cy5 dUTP (Amersham Pharmacia), each of the mRNA samples was reverse transcribed. The composition of the reverse transcription buffer (30 μl) is as follows.

poly (A)⁺ RNA with 6 μg oligo (dT) 18-mer 1 μg

10 mM DTT

500 μM dATP, dCTP and dGTP

200 μM dTTP

100 μM Cy3 dUTP or Cy5 dUTP

400 units of SuperScript II reverse transcription enzymes (Life Technologies)

1× Superscript first strand buffer (Life Technologies)

30 μl in total

After reaction at 42° C. for 1 hour, two samples (one sample labeled with Cy3 and the other sample labeled with Cy5) were mixed, and 15 μl of 0.1M NaOH and 1.5 μl of 20 mM EDTA were added thereto and the mixture was treated at 70° C. for 10 minutes. Then, 15 μl of 0.1M HCl was further added thereto, and the sample then transferred to a Micro con 30 micro concentrator (Amicon). 400 μl of TE buffer was added, followed by centrifugation so that the amount of the buffer became 10 to 20 μl, and then an effluent was thrown away. After 400 μl of TE buffer and 20 μl of 1 mg/ml human Cot-1 DNA (Gibco BRL) were added thereto, the mixture was subjected to centrifugation again. The labeled samples were collected by centrifugation, and several μl of distilled water were added thereto. To the obtained probes, 2 μl of 10 μg/μl yeast tRNA, 2 μl of 1 μg/μl pd(A)₁₂₋₁₈ (Amersham Pharmacia), 3.4 ml of 20×SSC and 0.6 μl of 10% SDS were added. Furthermore, the samples were denatured at 100° C. for 1 minute and placed at room temperature for 30 minutes, and then used for hybridization.

(9) Microarray Hybridization and Scanning

Using benchtop micro centrifuge, a probe was subjected to high-speed centrifugation for 1 minute. To avoid generation of bubbles, the probe was placed in the center of an array, and a cover slip was placed thereon. Four drops of 5 μl of 3×SSC were dropped on a slide glass and a chamber was kept at suitable humidity to prevent dehydration of the probe during hybridization. The slide glass was placed into a cassette for hybridization (THC-1, BM machine) and sealed, followed by treatment at 65° C. for 12 to 16 hours. The slide glass was taken out of the cassette and placed on a cassette rack, and a cover slip was carefully removed therefrom in solution 1 (2×SSC, 0.1% SDS). Thereafter, the rack was shaken to wash, and transferred into solution 2 (1×SSC) to wash for 2 minutes. Then, the rack was further transferred into solution 3 (0.2×SSC) and left for 2 minutes, and then subjected to centrifugation (800 rpm, 1 min) for drying.

Using a scanning laser microscope (ScanArray4000; GSI Lumonics, Watertown, Mass.), a microarray was scanned in a resolution of 10 μm per pixel. As a program for analyzing the microarray data, Imagene Ver 2.0 (BioDiscovery) and Quant Array (GSI Lumonics) were used.

(10) Northern Analysis

Using total RNA, Northern analysis was carried out (Yamaguchi-Shinozaki and Shinozaki, 1994). DNA fragments isolated from Arabidopsis thaliana full-length cDNA libraries by PCR were used as probes for Northern hybridization.

(11) Determination of Promoter Region

Based on the genomic information of Arabidopsis thaliana in database (GenBank/EMBL, ABRC), a promoter region was analyzed using the BLAST program for gene analysis.

2. Results

(1) Stress-Inducible Gene

Fluorescent-labeled cDNA was prepared from mRNA, which were isolated from unstressed Arabidopsis thaliana plants, by reverse transcription in the presence of Cy5-dUTP. From cold-treated plants (2 hours), the second probes labeled with Cy3-dUTP were prepared. Both types of probes were simultaneously hybridized to a cDNA microarray comprising about 1,300 Arabidopsis thaliana cDNA clones, and then a pseudo color image was created (FIG. 1).

Genes induced and inhibited by cold stress are represented by a red signal and a green signal, respectively. Genes which expressed at almost an equivalent level in both treatments are represented by a yellow signal. The strength of each spot corresponds to the absolute value of the expression level of each gene. It is shown that a cold-inducible gene (rd29A) is represented by a red signal whereas an α-tubulin gene (an internal control) is represented by a yellow signal.

By means of cDNA microarray analysis, the total 44 drought-inducible genes were identified (Tables 1 and 2). Table 1.

TABLE 1

| | Number of genes |
|---|---|
| Drought-inducible gene | 44 |
| New drought-inducible gene | 30 |
| Cold-inducible gene | 19 |
| New cold-inducible gene | 10 |
| DREB1A target gene | 12 |
| New DREB1A target gene | 6 |

TABLE 2
Drought- and Cold-inducible Genes, and DREB1A Target Genes Identified by cDNA Microarray Analysis

| | | | Drought (2 hr) | | Cold (2 hr) | | 35S:DREB1A | |
|---|---|---|---|---|---|---|---|---|
| | | | | New or | | New or | | New or |
| Gane | Accession | Coded Protein/Other Features | Ratio | Reported | Ratio | Reported | Ratio | Reported |
| rd29A | D13044 | Hydrophilic protein | 6.4 | Reported | 5.1 | Reported | 7.9 | Reported |
| cor15a | U01377 | — | 5.0 | Reported | 3.4 | Reported | 8.1 | Reported |
| kin2 | X55053 | — | 5.8 | Reported | 2.9 | Reported | 4.9 | Reported |
| erd10 | D17714 | Group II LEA protein | 6.0 | Reported | 4.6 | Reported | 3.5 | Reported |
| kin1 | X51474 | — | 2.9 | Reported | 2.0 | Reported | 3.4 | Reported |
| rd17 | AB004872 | Group II LEA protein | 6.4 | Reported | 4.6 | Reported | 4.6 | Reported |
| rd20 | — | Ca-binding EF hand protein | 5.1 | Reported | n.d. | — | n.d. | |
| erd7 | — | — | 3.8 | Reported | 2.3 | Reported | n.d. | |
| erd4 | — | Membrane protein | 2.6 | Reported | 2.2 | Reported | 2.5 | New |
| erd3 | — | — | 2.6 | Reported | n.d. | — | n.d | — |
| FL3-5I9 | D17715 | Group II LEA protein | 3.5 | Reported | 1.5 | — | 1.2 | — |
| FL3-3A1 | D13042 | Thiol protease | 2.8 | Reported | 1.9 | — | 1.5 | — |
| FL5-1F23 | D32138 | Δ¹-pyrroline-5-carboxylate synthetase (AtP5CS) | 2.8 | Reported | 1.5 | — | n.d. | — |
| FL2-1F6 | D01113 | Unidentified seed protein | 2.2 | Reported | 1.1 | — | 0.5 | — |
| FL3-5A3 | AC006438 | Putative cold acclimation protein | 6.2 | New | 2.3 | New | 3.4 | New |
| FL6-55 | X91919 | LEA 76 type 1 protein | 2.9 | New | n.d. | — | n.d. | — |
| FL5-77 | AF121355 | Peroxiredoxin TRX1 | 2.2 | New | 1.9 | — | 3.0 | New |
| FL3-5J1 | AF057137 | Gamma tonoplast intrinsic protein 2 (TIP2) | 2.0 | New | 1.2 | — | 1.3 | — |
| FL5-1N11 | M80567 | Non-specific lipid transfer protein (LTP1) | 2.7 | New | 1.3 | — | 1.2 | — |
| FL5-95 | — | Rice glyoxalase I homolog | 2.3 | New | 2.8 | New | n.d. | — |
| FL5-2H15 | T45998(EST) | — | 2.1 | New | n.d. | — | 1.4 | — |
| FL5-2O24 | AC005770 | Putative water channel protein | 2.4 | New | n.d. | — | 1.4 | — |
| FL5-2G21 | AF034255 | Reversibly glycosylated polypeptide-3 (RGP) | 2.1 | New | n.d. | — | 1.3 | — |
| FL5-1A9 | — | Nodulin-like protein homolog | 2.9 | New | 2.1 | New | 0.8 | — |
| FL5-94 | X58107 | Enolase | 2.0 | New | 1.8 | — | 2.3 | New |
| FL5-3J4 | — | Heat shock protein dnaJ homolog | 2.8 | New | 1.4 | — | n.d. | — |
| FL5-3M24 | — | LEA protein SAG21 homolog | 2.3 | New | 2.2 | New | 1.0 | — |
| FL5-1O3 | H37392(EST) | — | 3.0 | New | n.d. | — | n.d. | — |
| FL5-2I23 | D14442 | Ascorbate peroxidase | 2.1 | New | 1.1 | — | 1.0 | — |
| FL1-159 | AB015098 | HVA22 homolog | 3.7 | New | 3.8 | New | 1.9 | — |
| FL3-27 | — | Cysteine proteinase inhibitor homolog | 2.2 | New | n.d. | — | 2.2 | New |
| FL5-2I22 | X80342 | DC 1.2 homolog | 2.6 | New | 2.9 | New | 2.1 | New |
| FL5-1C20 | — | Major latex protein type I homolog | 2.0 | New | 1.4 | — | 1.8 | — |
| FL2-1H6 | — | Brassica napus jasmonate-inducible protein homolog | 2.4 | New | 1.3 | — | 0.9 | — |
| FL5-2E17 | — | Brassica napus beta-glucosidase homolog | 2.3 | New | 1.1 | — | 1.0 | — |
| FL3-3B1 | AC006403 | Hypothetical protein | 2.7 | New | 1.4 | — | 1.1 | — |
| FL5-3E18 | M80567 | Aquaporin homolog | 2.0 | New | 1.1 | — | 0.9 | — |
| FL5-3A15 | X94248 | Ferritin | 2.8 | New | 2.1 | New | 0.9 | — |
| FL2-56 | AF104330 | Glycine-rich protein 3 short isoform (GRP35) | 2.6 | New | 1.4 | — | 1.6 | — |
| FL5-2D23 | — | T20517 (EST) homolog | 2.6 | New | 1.3 | — | n.d. | — |
| FL3-2C6 | Z35474 | Thioredoxin | 2.3 | New | 1.0 | — | 1.9 | — |
| FL5-1P10 | AC004044 | Putative photosystem I reaction center subunit II precursor | 2.1 | New | 1.4 | — | 1.2 | — |
| FL2-5G7 | U43147 | Catalase 3 (CAT3) | 2.4 | New | 1.2 | — | 1.6 | — |
| FL2-1C1 | Z9734 | Cysteine proteinase homolog | 3.0 | New | 1.0 | — | 1.7 | — |
| DREB1A | AB007787 | EREBP/AP2 protein | n.d | — | 6.3 | Reported | 5.8 | — |
| FL2-5A4 | AB010259 | DEAD box ATPase/RNA helicase protein (DRH1) | n.d. | — | 2.1 | New | n.d. | — |
| FL5-90 | AJ250341 | β-amylase | n.d. | — | 4.4 | New | 1.2 | — |
| FL5-3P12 | D63510 | EXGT-A2 | 1.2 | — | 3.2 | New | 0.8 | — |

In Table 2, genes which are drought- and cold-inducible, and DREB1A target genes (35S:DREB1A) are rd29, cor15A, kin2, erd10, kin1, rd17, erd4, FL3-5A3, FL5-77, FL5-94, FL3-27 and FL5-2122. Genes which are drought- and cold inducible but are not DREB1A target genes, are FL5-2024, FL5-1A9, FL5-3M24 and FL5-3A15. Specifically drought-inducible genes are rd20, FL6-55, FL5-3J4, FL2-56 and FL5-2D23. Specifically cold-inducible genes are DREB LA and FL5-90. The results of classification of these genes are shown in FIG. 4.

Moreover, in Table 2, the “Coded Protein/Other Features” column shows putative functions of a gene product, which are predicted from sequence homology.

The “Ratio” in the “Drought” column is obtained by the following equation (FI: fluorescence intensity):
Ratio=[(the FI of each cDNA under drought condition)/(the FI of each cDNA under unstressed condition)]÷[(the FI of α-tubulin under drought condition)/(the FI of α-tubulin under unstressed condition)]

The “Ratio” in the “Cold” column is obtained by the following equation (FI: fluorescence intensity):
Ratio=[(the FI of each cDNA under cold condition)/(the FI of each cDNA under unstressed condition)]÷[(the FI of α-tubulin under cold condition)/(the FI of α-tubulin under unstressed condition)]

The “Ratio” in the “35S:DREB1A” column is obtained by the following equation (FI: fluorescence intensity):
Ratio=[(the FI of each cDNA of 35S:DREB1A plants)/(the FI of each cDNA of wild type plants)]÷[(the FI of α-tubulin of 35S:DREB1A plants)/(the FI of α-tubulin of wild type plants)]

With regard to the term “New or Reported” in the “Drought” column, where a gene has not been reported as a drought-inducible gene, it is shown as “New”, and where it has been reported as a drought-inducible gene, it is shown as “Reported”. The same applies for a cold-inducible gene in the “Cold” column and a DREB1A target gene in the “35S:DREB1A” column.

Among the genes described in Table 2, 14 genes (cor15A, kin1, kin2, rd17, rd19A, rd20, rd22, rd29A, erd3, erd4, erd7, erd10, erd14, AtP5CS) have previously been reported as drought-inducible genes (Bohnert, H. J. et al., (1995) Plant Cell 7, 1099-1111; Ingram, J., and Bartels, D. (1996) Plant Mol. Biol. 47, 377-403; Bray, E. A. (1997) Trends Plant Sci. 2, 48-54; Shinozaki. K., and Yamaguchi-Shinozaki, K. (1997) Plant Physiol. 115, 327-334; Shinozaki, K., and Yamaguchi-Shinozaki, K. (1999). Molecular responses to drought stress. Molecular responses to cold, drought, heat and salt stress in higher plants. Edited by Shinozaki, K. and Yamaguchi-Shinozaki, K., R. G. Landes Company; Shinozaki, K., and Yamaguchi-Shinozaki, K. (2000) Curr. Opin. Plant Biol. 3, 217-223; Taji, T. et al., (1999) Plant Cell Physiol. 40, 119-123; Takahashi, S. et al., (2000) Plant Cell Physiol. 41, 898-903.)

From these results, it is shown that the cDNA microarray system of the present inventors has functioned appropriately to find stress-inducible genes. In the remaining 30 new drought-inducible genes, there were found cDNA molecules (FL3-5A3, FL6-55, FL5-1N11, FL5-2024, FL5-2H15 and FL1-159) which show sequence identity with putative cold acclimation protein (Accession No. AC006438), LEA 76 type 1 protein (Accession No. X91919), non-specific lipid transfer protein (LTP1; Accession No. M80567) putative water channel protein (Accession No. AC005770), T45998 EST, and HVA22 homolog (Accession No. AB015098).

Moreover, the total 19 cold-inducible genes have been identified by cDNA microarray analysis (Tables 1 and 2). Nine of these genes have been reported as cold-inducible genes, rd29A, cor15a, kin1, kin2, rd17, erd10, erd7 and erd4 (Kiyosue et al., 1994; Shinozaki and Yamaguchi-Shinozaki, 1997, 1999, 2000; Taji et al., 1999; Thomashow, 1999) and a DREB1A gene (Lui et al., 1998).

Likewise, in the remaining new cold-inducible genes, there were found cDNA molecules (FL3-5A3, FL5-3A15, FL5-3P12, FL5-90, FL5-2122 and FL1-159) which show sequence identity with putative cold acclimation protein (Accession No. AC006438), ferritin (Accession No. X94248), EXGT-A2 (Accession No. D63510), 3-amylase (Accession No. AJ250341), DC 1.2 homolog (Accession No. X80342), and HVA22 homolog (Accession No. AB015098), and also found cDNA molecules (FL5-1A9, FL5-95 and FL5-3M24) which show sequence similarity with Nodulin-like protein (Accession No. CAA22576), rice glyoxalase I (Accession No. AB017042) and LEA protein homolog (SAG21; Accession No. AF053065).

Furthermore, the present inventors have identified stress-inducible genes controlled by a DREB1A transcription factor, using a full-length cDNA microarray. FIG. 2 shows a procedure for identification of DREB1A target genes. The mRNA prepared from a transgenic Arabidopsis thaliana plant (a 35S:DREB1A transgenic plant), which overexpresses DREB1A cDNA under the control of a CaMV 35S promoter, and the mRNA prepared from a wild type control plant were used to prepare Cy3-labeled and Cy5-labeled cDNA probes, respectively. These cDNA probes were mixed and then hybridized to a cDNA microarray. A gene having more than a two-fold greater expression level in a 35S:DREB1A transgenic plant than in a wild type control plant was defined as a DREB1A target gene.

The total 12 DREB1A target genes have been identified by cDNA microarray analysis (Tables 1 and 2). Six of these genes have been reported as DREB1A target genes, rd29A/cor78, cor15a, kin1, kin2, rd17/cor47 and erd10 Kasuga et al., 1999). Likewise, in the remaining 6 new DREB1A target genes, there were found cDNA molecules (FL3-5A3, FL5-2I22, FL5-94 and FL5-77) which show sequence identity with putative cold acclimation protein (Accession No. AC006438), DC 1.2 homolog (Accession No. X80342), enolase (Accession No. X58107) and peroxiredoxin TPX1 (Accession No. AF121355), and also found a cDNA molecule (FL3-27) showing sequence similarity with a cowpea cysteine proteinase inhibitor (Accession No. Z21954) and erd4 cDNA (Kiyosue et al., 1994; Taji et al., 1999).

The identified drought- or cold-inducible genes were classified into the following 3 groups (FIG. 4):

1) Drought- and cold-inducible genes

2) Specifically drought-inducible genes

3) Specifically cold-inducible genes

With regard to the following 21 genes, it was difficult to classify them into the above 3 groups and so they did not undergo such a classification: erd3, FL3-519, FL3-3A1, FL5-1F23, FL2-1F6, FL3-5J1, FL5-1N11, FL5-2H15, FL5-2G21, FL5-2123, FL5-1C20, FL2-1H6, FL5-2E17, FL3-3B1, FL5-3E18, FL3-2C6, FL5-1P10, FL2-5G7, FL2-1C1, FL2-5A4, and FL5-3P12

As a result, the identified genes were classified into 20 drought- and cold-inducible genes, 5 specifically drought-inducible genes and 2 specifically cold-inducible genes. Thereafter, the drought- and cold-inducible genes were classified into two groups:

1) DREB1A target genes

2) Genes other than DREB1A target genes

Thus, 16 drought- and cold-inducible genes were classified into 12 DREB1A target genes and 4 genes other than DREB1A target genes.

(2) RNA Gel Blot Analysis

In cDNA gel blot analysis, it is important to extract appropriate data with minimum effort. The present inventors evaluated the effectiveness of cDNA microarray analysis by the following method.

First, 80 genes having more than double the expression ratio (drought 2 hours/unstressed) of α-tubulin were identified. The 80 putative drought-inducible genes were subjected to Northern blot analysis, 44 of which were identified as actual genes. The disparity between the results from microarray analysis and those from Northern blot analysis was caused by (1) low expression of genes, (2) high background, (3) dusts or scratches on a cDNA spot, and (4) a bad cDNA probe with a low specific activity. Accordingly, the above experimental data were marked and a half thereof was excluded from the following analysis. After the data processing, 44 drought-inducible genes, 19 cold-inducible genes and 12 DREB1A target genes were finally identified based on cDNA microarray analysis. Thereafter, RNA gel blot analysis was carried out to confirm the obtained results using a cDNA microarray. The result of expression data obtained by microarray analysis that 44 drought-inducible genes, 19 cold-inducible genes and 12 DREB1A target genes were identified, was consistent with the result obtained by Northern blot analysis.

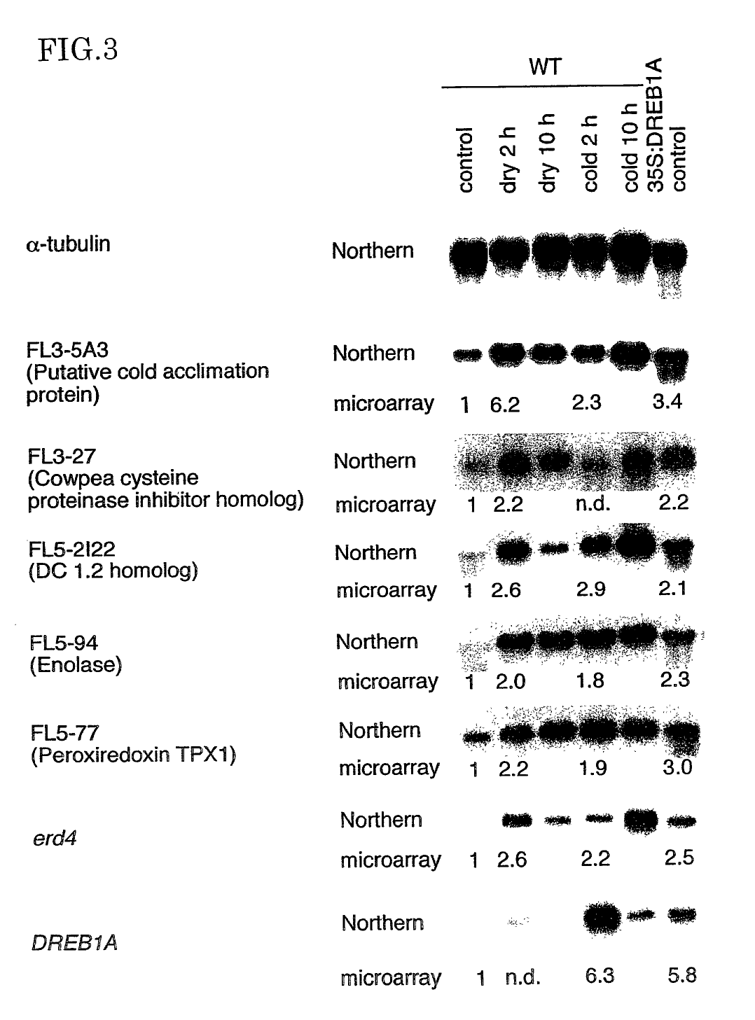

FIG. 3 shows a comparison between the result of microarray analysis and that of Northern blot analysis in respect of 6 new DREB1A target genes (FL3-5A3, FL3-27, FL5-2I22, FL5-94, FL5-77 and erd4). All of the 6 genes were induced by drought- and cold-treatments and overexpressed in 35S:DREB1A plants under unstressed conditions.

Samples derived from drought-treated wild type plants (dehydration for 2 hours or 10 hours (left on a filter paper)), cold-treated wild type plants (cooling at 4° C. for 2 hours or 10 hours), or untreated 35S:DREB1A transgenic plants (35S:DREB1A control) were subjected to fluorescent labeling with Cy3-dUTP, whereas samples derived from untreated wild type plants (control) were subjected to fluorescent labeling with Cy5-dUTP. These samples were hybridized to a cDNA microarray followed by scanning to calculate a relative expression ratio and shown in a figure (FIG. 3). The figure shows Northern blot analysis images of drought- and cold-treated wild type plants and 35S:DREB1A transgenic plants. The full-length cDNA sequences of two DREB1A target genes (FL3-5A3 and FL3-27) are independently registered with GenBank, EMBL and DDBJ database under Accession Nos. AB044404 and AB044405, respectively.

Likewise, using a full-length cDNA microarray containing about 7,000 Arabidopsis full-length cDNA molecules, 301 drought-inducible genes, 54 cold-inducible genes and 211 high salt stress-inducible genes were isolated.

(3) Identification of Promoter Region

As a result of identification of promoter regions, there were obtained the promoter regions of 8 types of genes (FL3-5A3, FL5-2H15, FL5-3M24, FL5-90, FL5-2I22, FL6-55, FL1-159 and FL5-2D23) obtained in a full-length cDNA microarray containing about 1,300 Arabidopsis full-length cDNA molecules. The sequences of these promoters are shown in SEQ ID NOS: 1 to 8.

| Gene Name | Promoter Region Sequence |
|---|---|
| FL3-5A3 | SEQ ID NO: 1 |
| FL5-2H15 | SEQ ID NO: 2 |
| FL5-3M24 | SEQ ID NO: 3 |
| FL5-90 | SEQ ID NO: 4 |
| FL5-2I22 | SEQ ID NO: 5 |
| FL6-55 | SEQ ID NO: 6 |
| FL1-159 | SEQ ID NO: 7 |
| FL5-2D23 | SEQ ID NO: 8 |

As a result of the identification of promoter regions, there were obtained the promoter regions of 10 types of genes (FL05-08-P24, FL05-09-G08, FL05-09-P10, FL05-10-NO₂, FL05-18-I12, FL05-21-F13, FL06-10-C16, FL06-15-P15, FL08-10-E21 and FL09-11-P10) obtained in a full-length cDNA microarray containing about 7,000 Arabidopsis full-length cDNA molecules. The sequences of these promoters are shown in SEQ ID NOS: 9 to 18.

| Gene Name | Promoter Region Sequence |
|---|---|
| FL05-08-P24 | SEQ ID NO: 9 |
| FL05-09-G08 | SEQ ID NO: 10 |
| FL05-09-P10 | SEQ ID NO: 11 |
| FL05-10-N02 | SEQ ID NO: 12 |
| FL05-18-I12 | SEQ ID NO: 13 |
| FL05-21-F13 | SEQ ID NO: 14 |
| FL06-10-C16 | SEQ ID NO: 15 |
| FL06-15-P15 | SEQ ID NO: 16 |
| FL08-10-E21 | SEQ ID NO: 17 |
| FL09-11-P10 | SEQ ID NO: 18 |

It is known that a conserved sequence “PyACGTG (G or T)C” (wherein Py represents a pyrimidine base, that is, C or T) acts as an ABA-responsive element (ABRE) in many ABA-responsive genes (Ingram, J., and Bartels, D. (1996) Plant Mol. Biol. 47, 377-403; Bray, E. A. (1997) Trends Plant Sci. 2, 48-54; Shinozaki, K., and Yamaguchi-Shinozaki, K. (1999). Molecular responses to drought stress. Molecular responses to cold, drought, heat and salt stress in higher plants. Edited by Shinozaki, K. and Yamaguchi-Shinozaki, K., R. G. Landes Company.) Furthermore, it is also known that a conserved sequence consisting of 9 nucleotides “TACCGACAT” (a dehydration-responsive element: DRE) is essential to the induction control of rd29A expression under drought-, cold- and high salt-stress conditions, but does not function as an ABA-responsive element (ABRE) (Yamaguchi-Shinozaki, K., and Shinozaki, K. (1994) Plant Cell 6, 251-264). It is also known that CRT or LTRE, which is DRE or DRE-related core motifs (CCCAG), exists in the regions of drought- and cold-inducible genes (e.g. kin1, kin2, rd17/cor47 and cor15a) (see Table 3, Baker, S. S. et al., (1994) Plant Mol. Biol. 24, 701-713; Wang, H. et al., (1995) Plant Mol. Biol. 28, 605-617; Iwasaki, T. et al., (1997) Plant Physiol. 115, 1287.)

TABLE 3

ABRE, DRE, and CCGAC Core Sequences Observed in the
Promoter Regions of the DREB1A Target Genes Identi-
fied by cDNA Microarray Analysis a)

| | ABRE | DRE | CCGAC Core Motif |
|---|---|---|---|
| Gene | (PyACGTG(T/G)C) | (TACCGACAT) | (CCGAC) |
| rd29A | TACGTGTC(−45 to −38)^{b)} | TACCGACAT(−148 to −140) | AGCCGACAC(−111 to −103) |
| | | TACCGACAT(−206 to −198) | GACCGACTA(−256 to −248) |
| cor15A | CACGTGGC(−132 to −125) | — | GGCCGACCT(−184 to −176) |
| | | | GGCCGACAT(−361 to −353) |
| | | | AACCGACAA(−416 to −424) |
| kin1 | — | TACCGACAT(−120 to −112) | ATCCGAGAT(−720 to −712) |
| kin2 | CACGTGGC(−68 to −61) | TACCGACAT(−127 to −119) | CCCCGACGC(−403 to −395) |
| rd17 | TACGTGTC(−920 to −913) | — | TACCGACTT(−162 to −154) |
| | | | AGCCGACCA(−967 to −959) |
| | | | GACCGACAT(−997 to −989) |
| erd10 | CACGTGGC(−838 to −831) | — | GACCGACGT(−198 to −190)^{c)} |
| | | | GACCGACCG(−202 to −194)^{c)} |
| | | | CACCGACCG(−206 to −198)^{c)} |
| | | | GACCGACAT(−999 to −991) |
| FL3-5A3 | CACGTGGC(−74 to −67) | TACCGACAT(−415 to −407) | TGCCGACAT(−806 to −798) |
| FL3-27 | — | TACCGACAT(−89 to −81) | — |
| FL5-2I22 | — | — | TACCGACTC(−191 to −183) |
| | | | TACCGACTA(−266 to −258) |
| | | | TGCCGACCT(−418 to −410) |
| | | | ACCCGACTA(−695 to −703) |
| | | | GACCGACGT(−786 to −778) |
| FL5-77 | — | — | CCCCGACTA(−315 to −307) |
| FL5-94 | — | — | TACCGACTA(−190 to −198) |
| | | | TTCCGACTA(−260 to −268) |
| | | | ATCCGACGC(−630 to −622) |

In Table 3, a), b) and c) are defined as follows:

a): ABRE, DRE and CCGAC core sequences refer to sequences observed at 1,000 bp upstream of the 5′-terminus of the longest cDNA isolated.

b): Figures in parentheses represent nucleotides initiating at the 5′-termini of the isolated cDNA. A minus sign means that a nucleotide exists upstream of the 5′-terminus of a putative transcription initiation site.

c): Each of these sequences consisting of 9 nucleotides comprises a CCGAC core motif, and the sequences overlap with one another.

The present inventors identified 12 DREB1A target genes by cDNA microarray analysis. A DRE sequence of 9 bp is observed in the promoter regions of genes corresponding to the cDNA molecules of FL3-5A3 and FL3-27 (Table 3). A core, sequence “CCGAC” is observed in the promoter regions of genes corresponding to the cDNA molecules of FL3-5A3, FL5-2I22, FL5-77 and FL5-94 (Table 3). Almost all of the drought- and cold-inducible genes are DREB1A target genes, each of which contains a DRE/CRT cis-acting element in a promoter thereof (Table 3 and FIG. 4). An ABRE sequence (“PyACGTG(G or T)C”) was observed in 6 promoter regions among the identified 12 DREB1A target genes (Table 3). This shows that many drought- and cold-inducible genes are controlled by both ABA-dependent and ABA-independent routes. However, some drought- and cold-inducible genes (FL5-3M24, FL5-3A15, FL5-1A9 and FL5-2024) did not increase in 35S:DREB1A transgenic plants (FIG. 4). This shows that these genes are not DREB1A target genes. A core sequence “CCGAC” was not observed in a region 2,000 bp upstream of the 5′-terminus of cDNA of FL5-3M24. This result shows a possibility that a new cis-acting element associated with expression of drought- and cold-inducible genes exists in the promoter region of a FL5-3M24 gene.

(4) Relation Between Various Stress Treatment Periods of Time and Expression Ratio

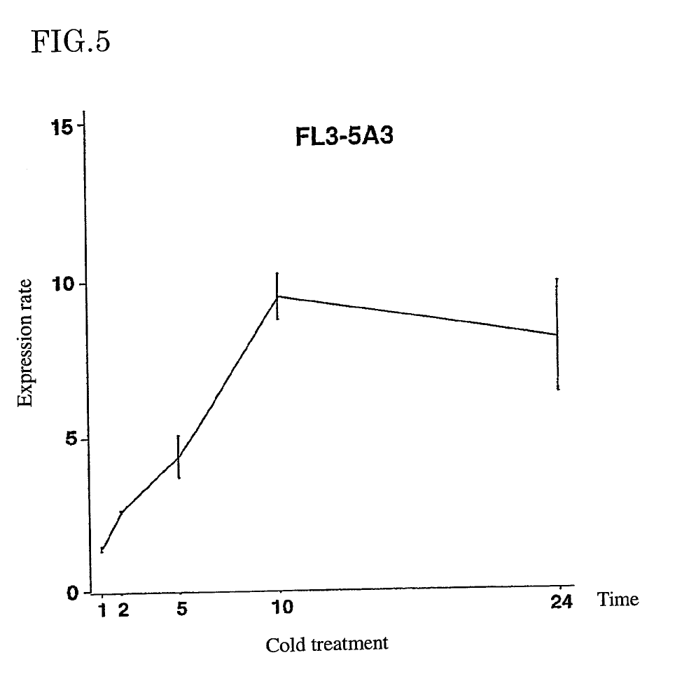

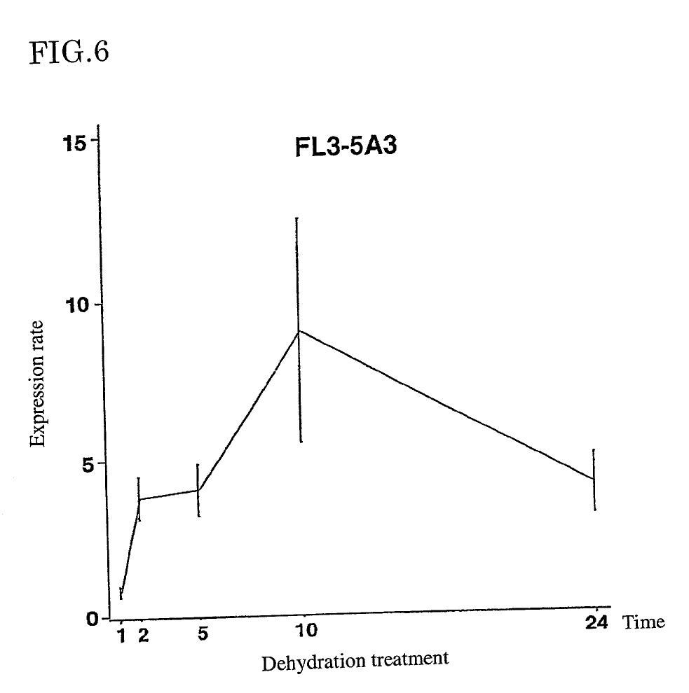

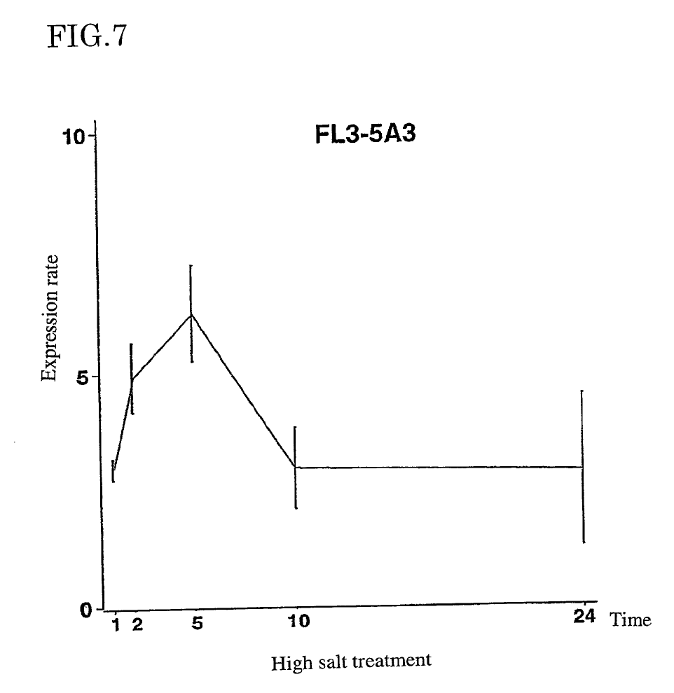

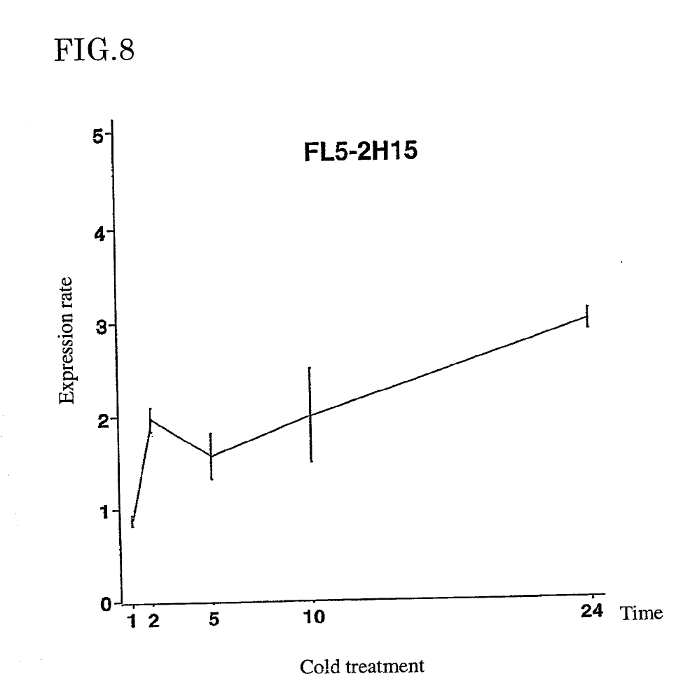

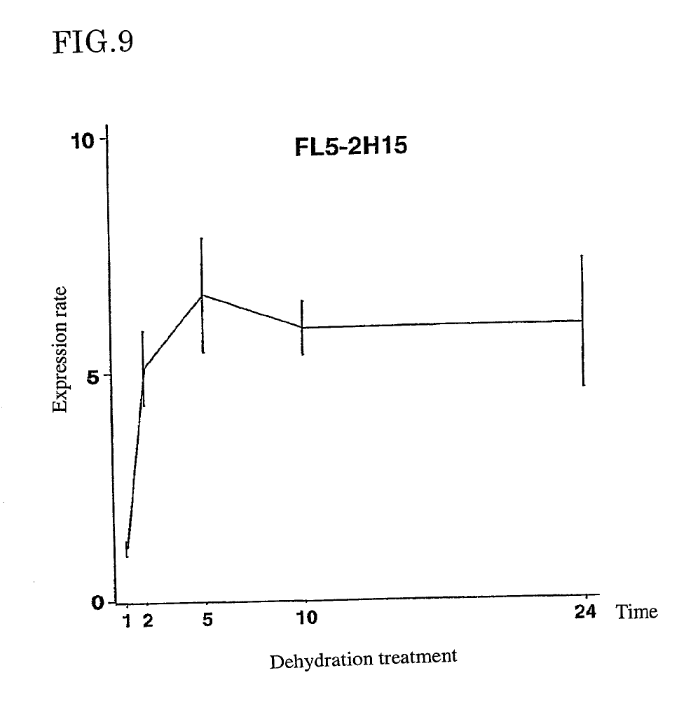

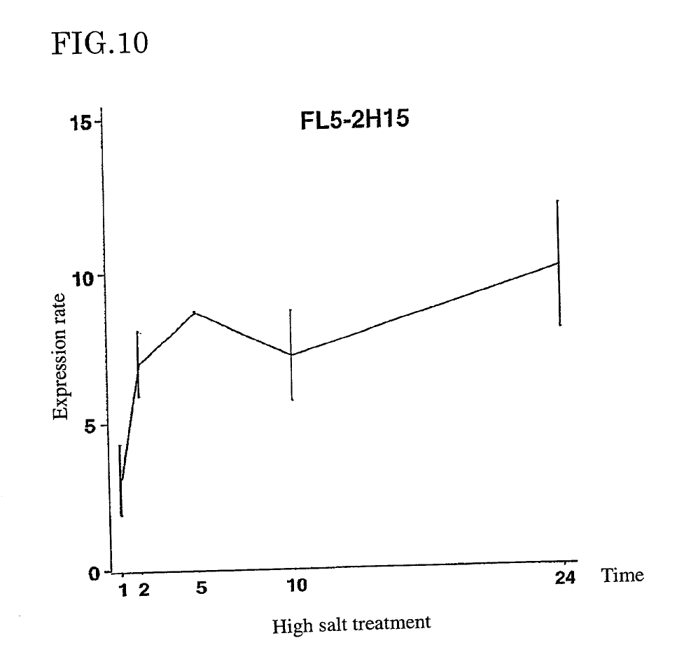

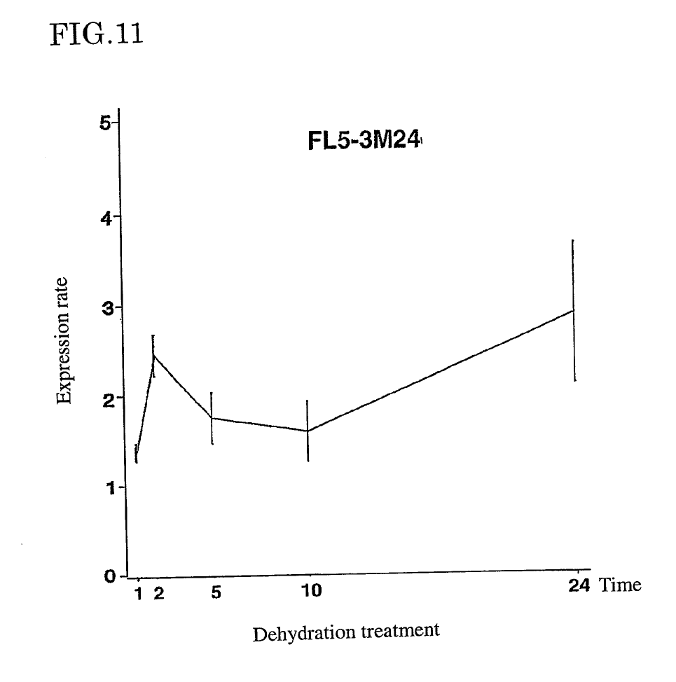

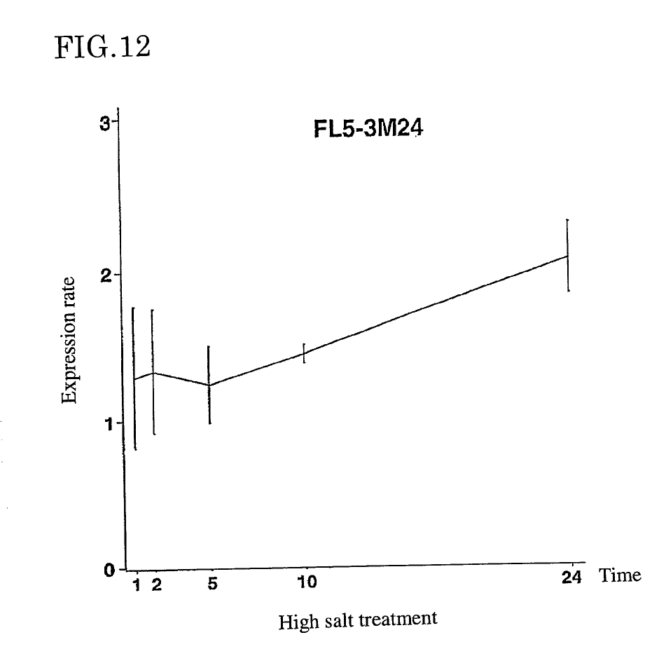

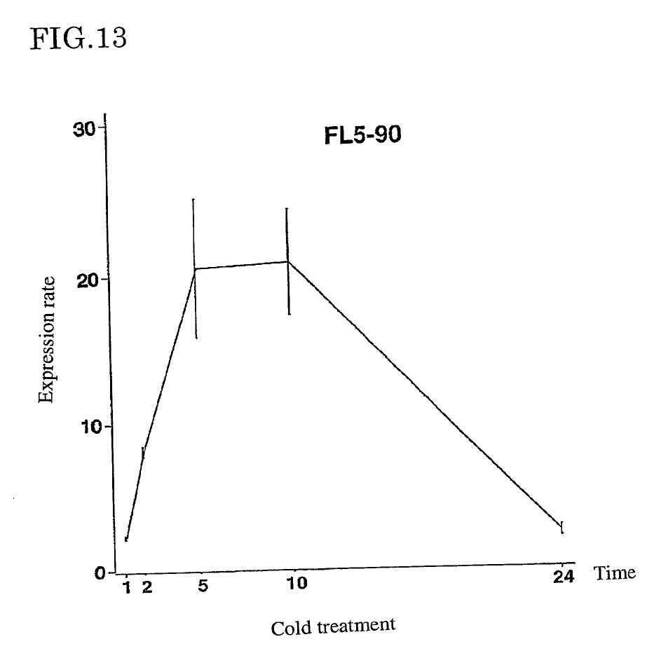

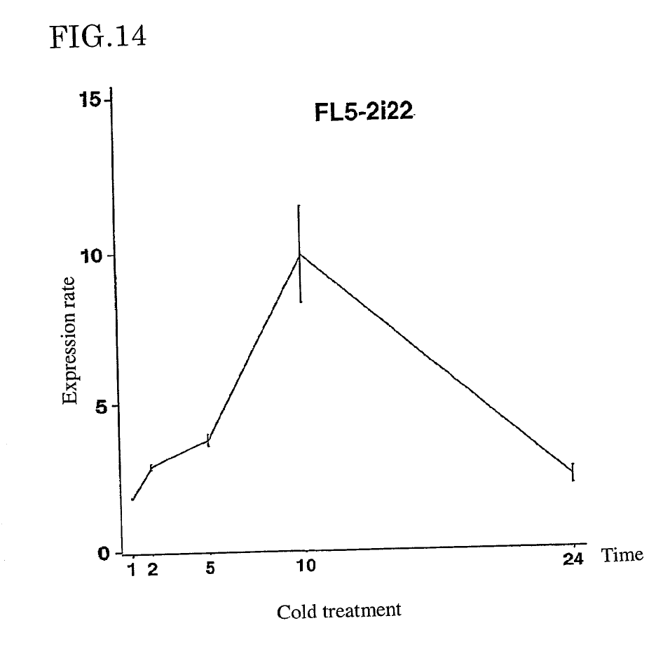

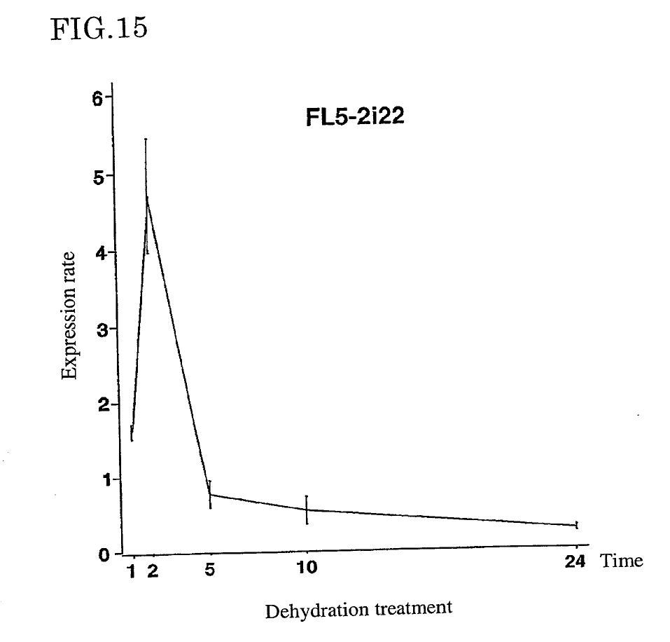

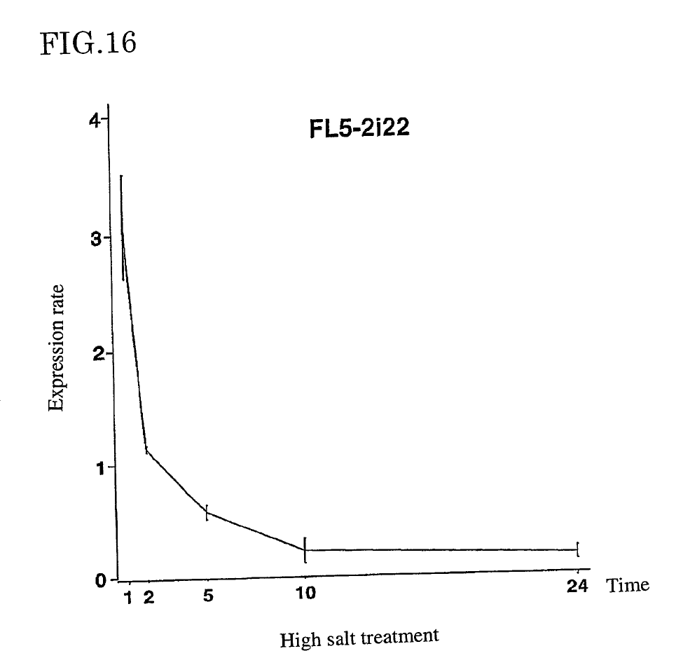

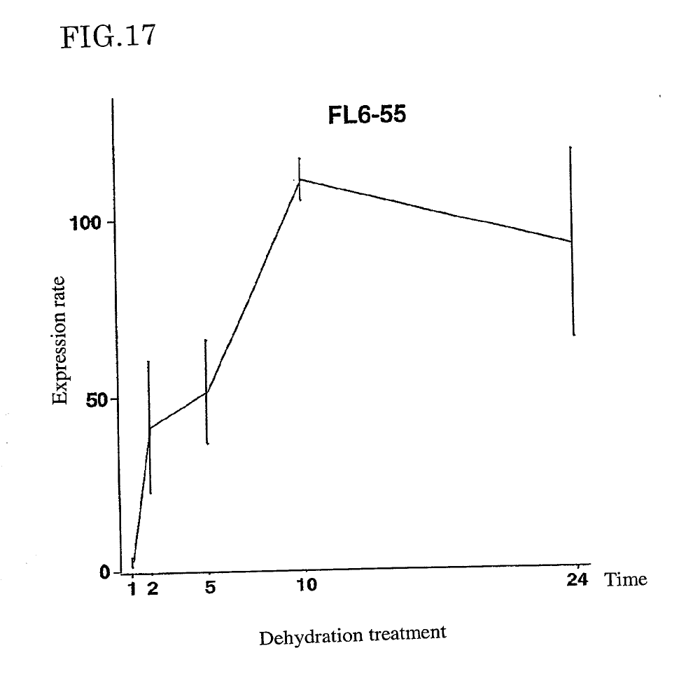

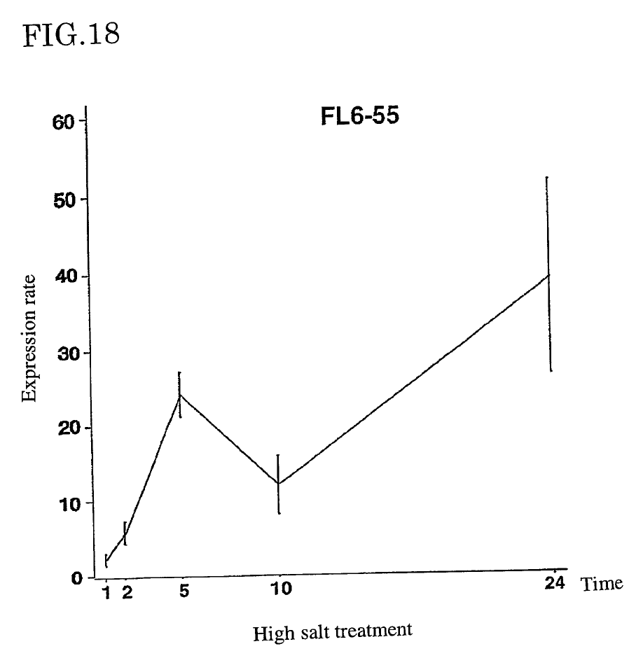

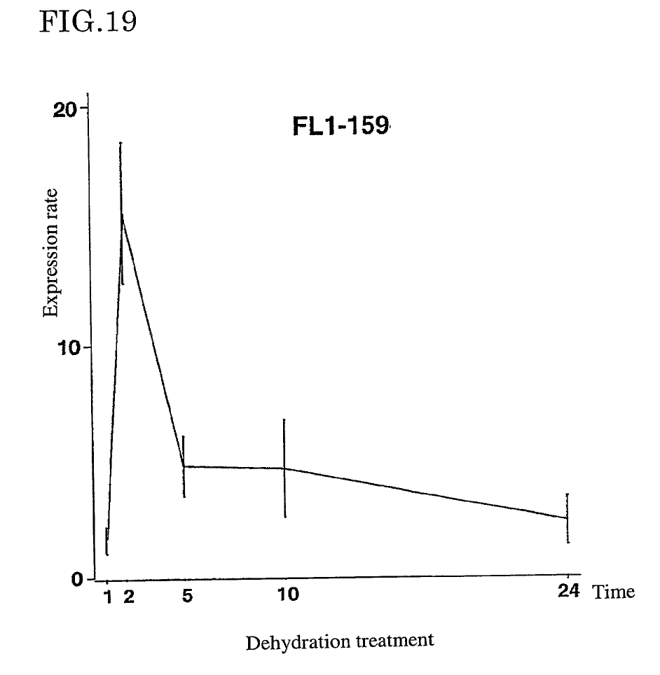

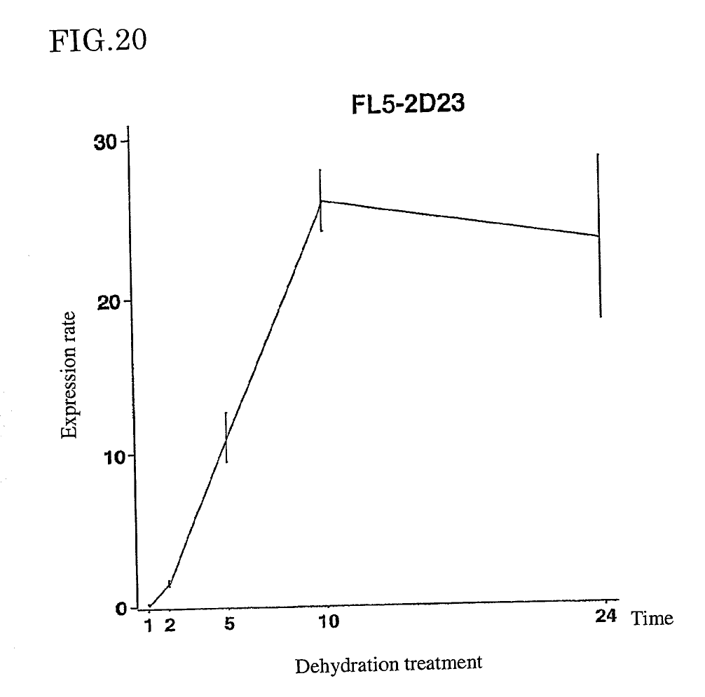

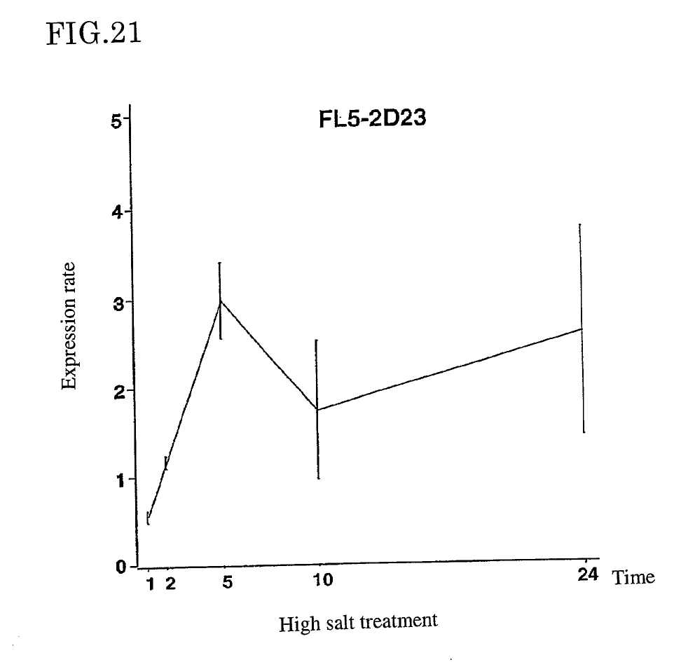

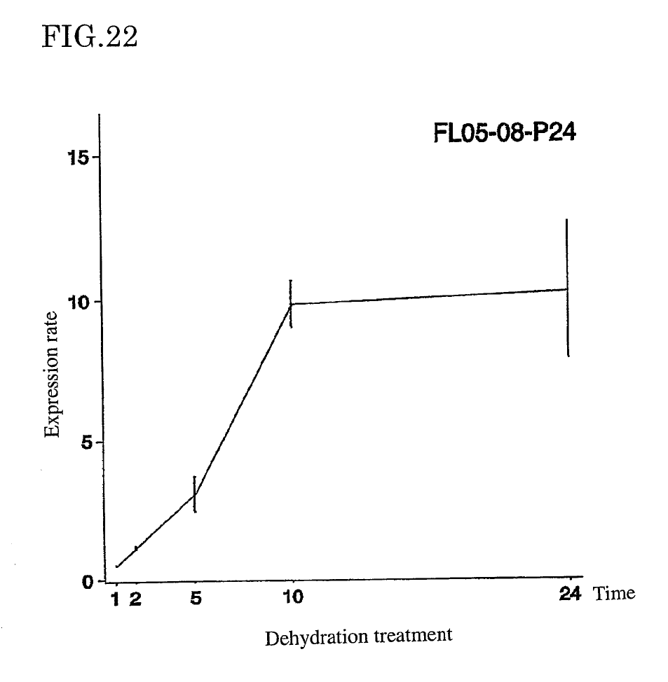

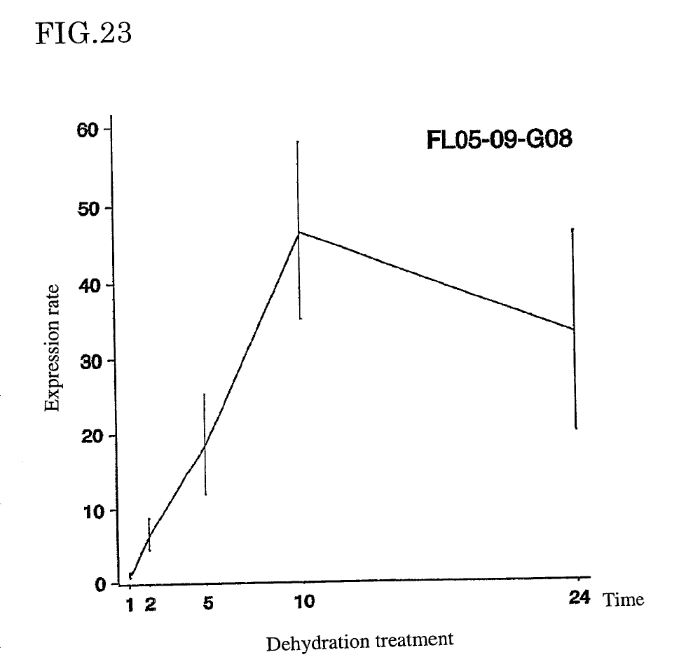

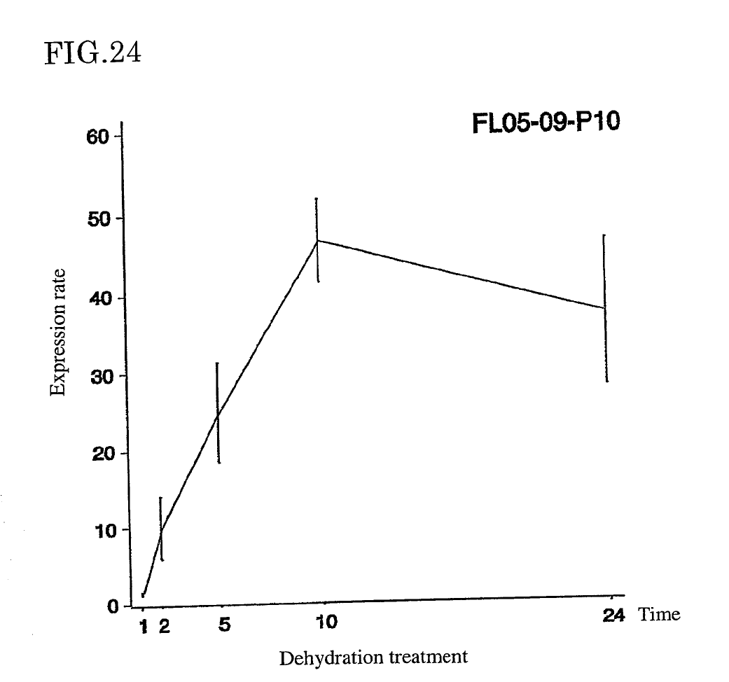

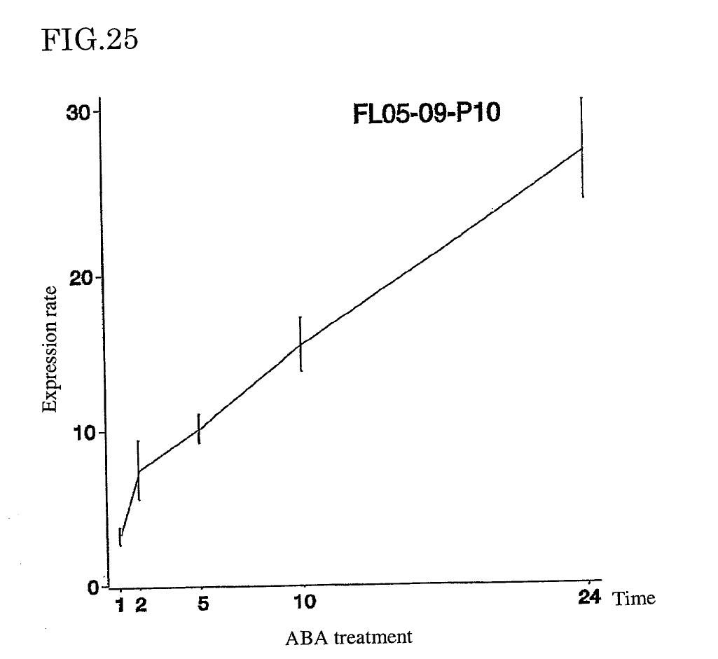

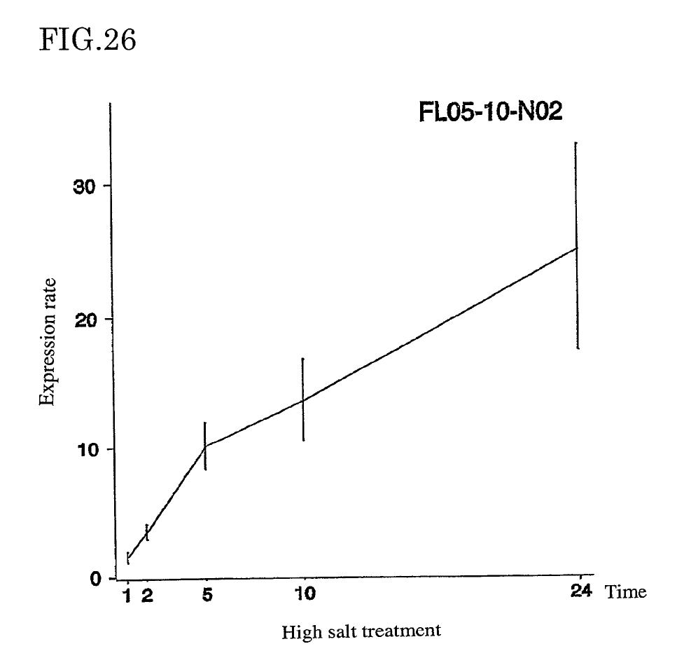

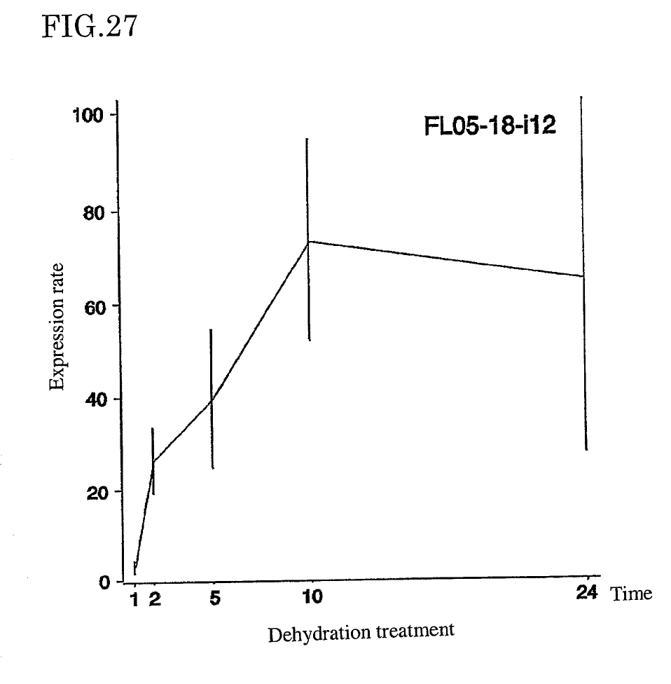

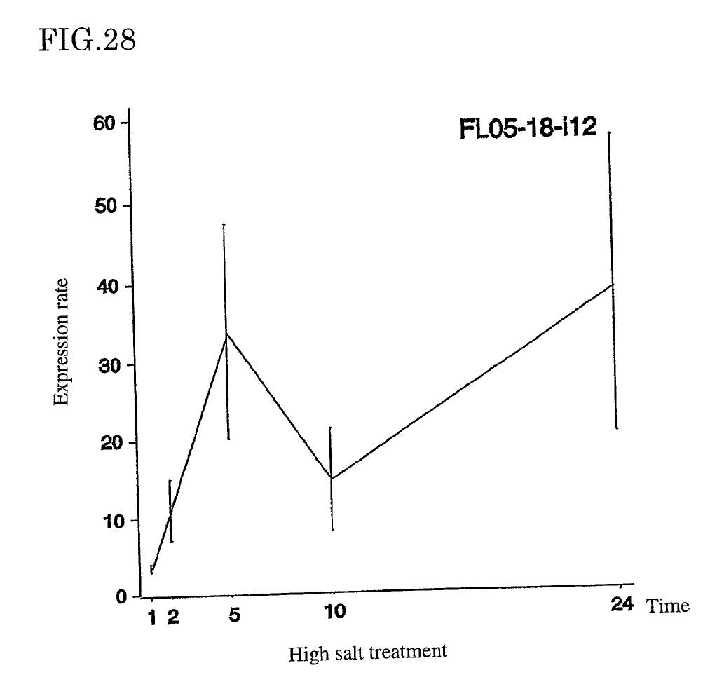

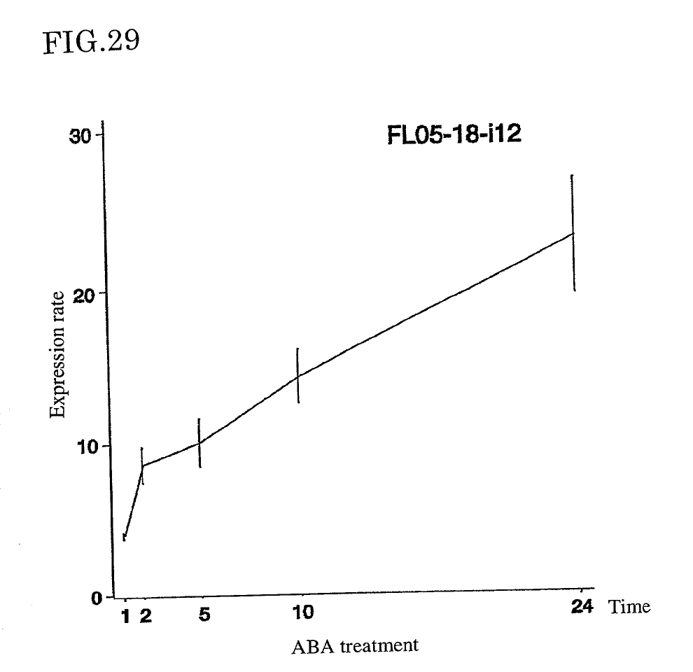

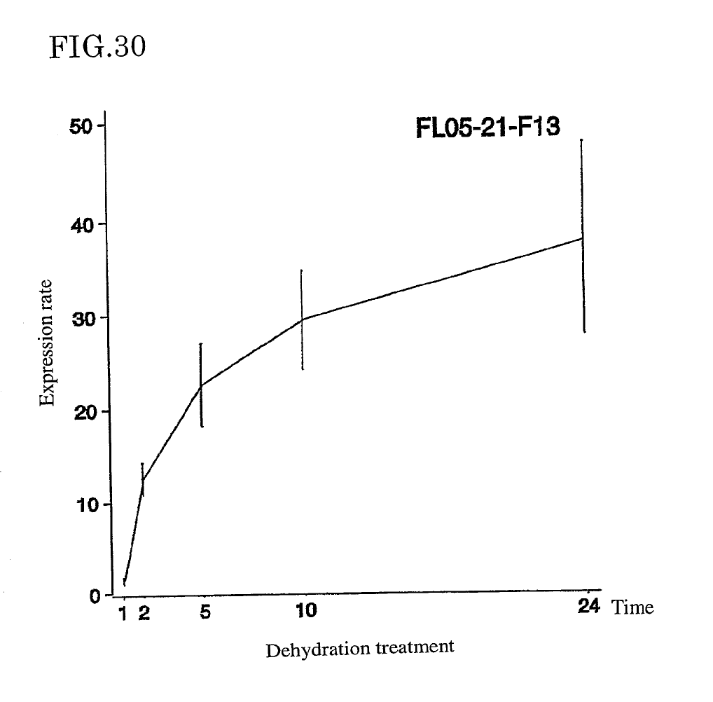

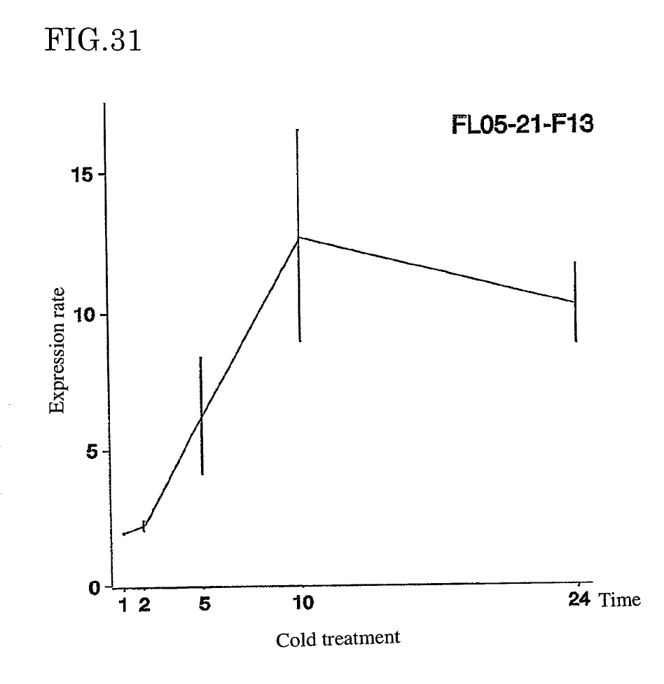

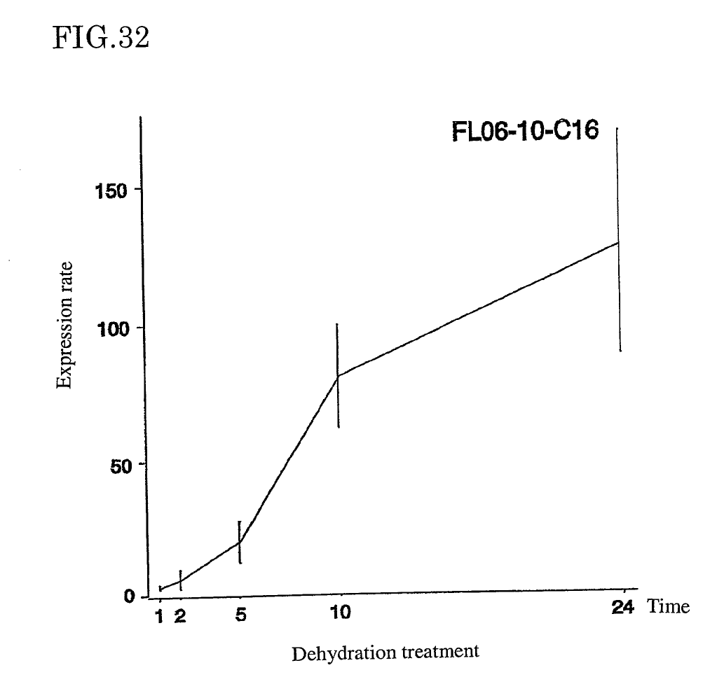

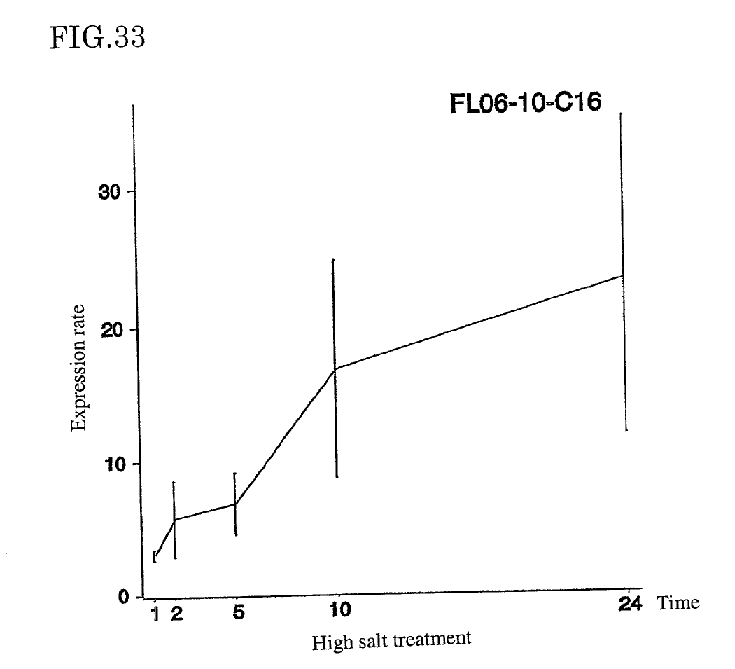

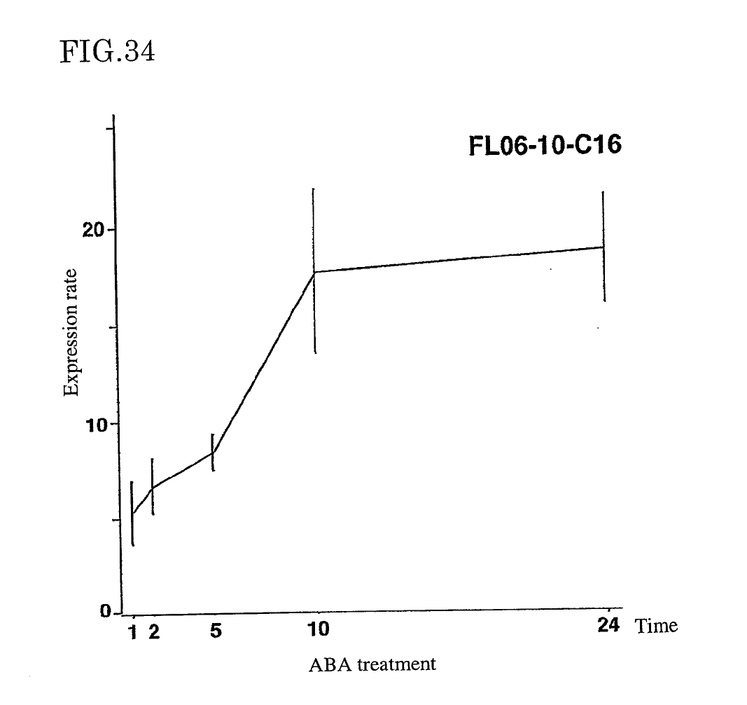

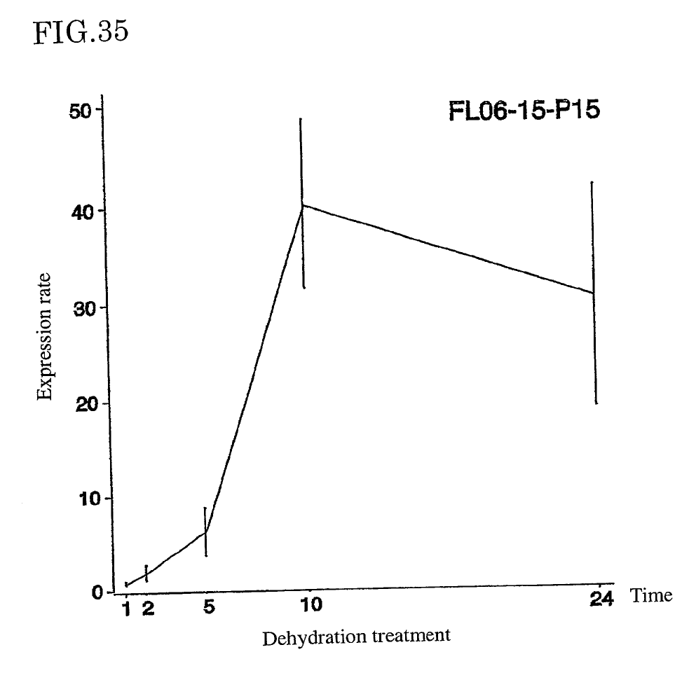

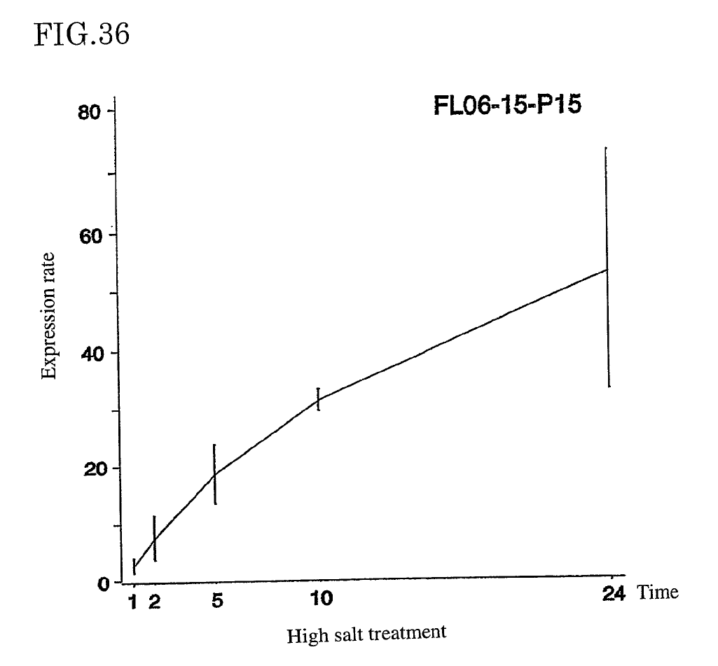

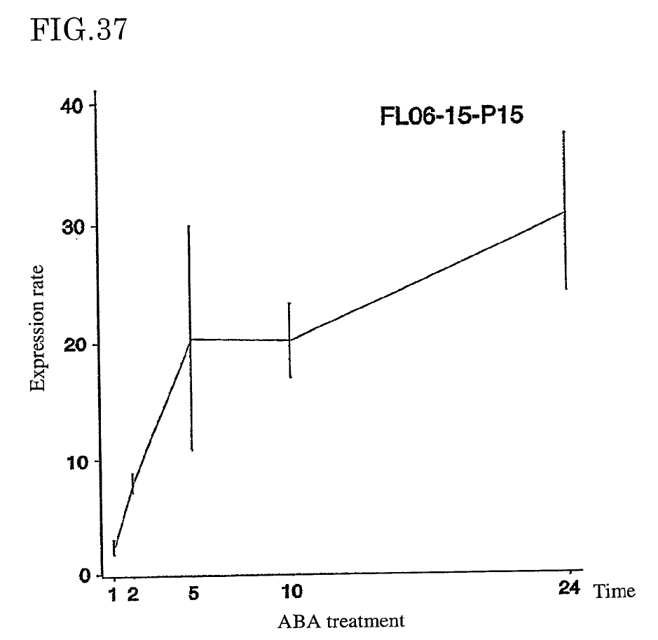

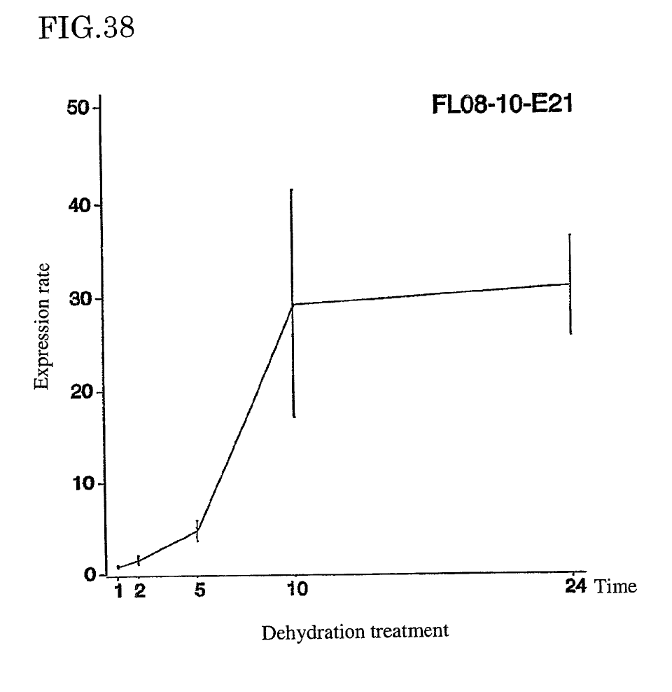

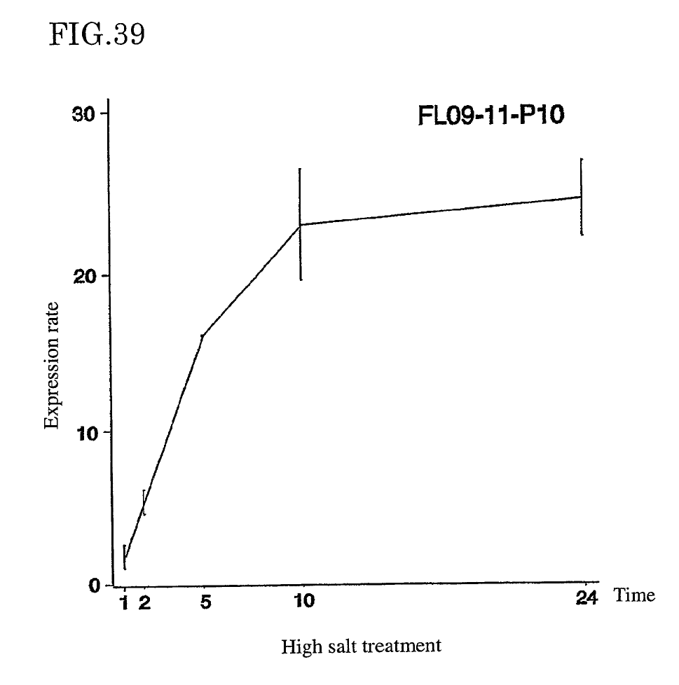

With regard to 18 types of stress-inducible genes isolated as above, the results of analysis of the relation between various stress treatment periods of time and expression ratio are shown in the following FIGS. 5 to 39.

| Gene Name | Stress Result (Figures showing the results) |
|---|---|
| FL3-5A3 | Cold (FIG. 5), Drought (FIG. 6) and High |
| | Salt (FIG. 7) |
| FL5-2H15 | Cold (FIG. 8), Drought (FIG. 9) and High |
| | Salt (FIG. 10) |
| FL5-3M24 | Drought (FIG. 11) and High Salt (FIG. 12) |
| FL5-90 | Cold (FIG. 13) |
| FL5-2I22 | Cold (FIG. 14), Drought (FIG. 15) and High |
| | Salt (FIG. 16) |
| FL6-55 | Drought (FIG. 17) and High Salt (FIG. 18) |
| FL1-159 | Drought (FIG. 19) |
| FL5-2D23 | Drought (FIG. 20) and High Salt (FIG. 21) |
| FL05-08-P24 | Drought (FIG. 22) |
| FL05-09-G08 | Drought (FIG. 23) |
| FL05-09-P10 | Drought (FIG. 24) and ABA (FIG. 25) |
| FL05-10-N02 | High Salt (FIG. 26) |
| FL05-18-I12 | Drought (FIG. 27), High Salt (FIG. 28) and |
| | ABA (FIG. 29) |
| FL05-21-F13 | Drought (FIG. 30) and Cold (FIG. 31) |
| FL06-10-C16 | Drought (FIG. 32), High Salt (FIG. 33) and |
| | ABA (FIG. 34) |
| FL06-15-P15 | Drought (FIG. 35), High Salt (FIG. 36) and |
| | ABA (FIG. 37) |
| FL08-10-E21 | Drought (FIG. 38) |
| FL-9-11-P10 | High Salt (FIG. 39) |

As shown in FIGS. 5 to 39, the stress-inducible genes isolated by the method of the present invention are different profiles, but are expressions induced by the addition of various types of stresses.

Effect of the Invention

According to the present invention, a stress responsive promoter is provided. The promoter of the present invention is useful in that it can be used for molecular breeding of environmental stress-resistant plants.

1. An environmental stress responsive promoter comprising DNA of the following (a), (b) or (c):
(a) DNA consisting of any nucleotide sequence selected from SEQ ID NOS: 1 to 8;
(b) DNA consisting of a nucleotide sequence comprising a deletion, substitution or addition of one or more nucleotides relative to any nucleotide sequence selected from SEQ ID NOS: 1 to 8, and functioning as an environmental stress responsive promoter; and
(c) DNA hybridizing under stringent conditions to DNA consisting of any nucleotide sequence selected from SEQ ID NOS: 1 to 8, and functioning as an environmental stress responsive promoter.

2. An environmental stress responsive promoter comprising DNA of the following (a), (b) or (c):
(a) DNA consisting of any nucleotide sequence selected from SEQ ID NOS: 9 to 18;
(b) DNA consisting of a nucleotide sequence comprising a deletion, substitution or addition of one or more nucleotides relative to any nucleotide sequence selected from SEQ ID NOS: 9 to 18, and functioning as an environmental stress responsive promoter; and
(c) DNA hybridizing under stringent conditions to DNA consisting of any nucleotide sequence selected from SEQ ID NOS: 9 to 18, and functioning as an environmental stress responsive promoter.

3. The promoter according to claim 1, wherein the environmental stress is at least one selected from the group consisting of cold stress, drought stress, salt stress and high photo stress.

4. An expression vector comprising the promoter according to claim 1.

5. The expression vector according to claim 4, into which a desired gene is further incorporated.

6. A transformant comprising the expression vector according to claim 4.

7. A transgenic plant comprising the expression vector according to claim 4.

8. The transgenic plant according to claim 7, wherein the plant is a plant body, plant organ, plant tissue or plant culture cell.

9. A method for producing a stress-resistant plant, which comprises culturing or cultivating the transgenic plant according to claim 7.

10. The promoter according to claim 2, wherein the environmental stress is at least one selected from the group consisting of cold stress, drought stress, salt stress and high photo stress.

11. An expression vector comprising the promoter according to claim 2.

12. An expression vector comprising the promoter according to claim 3.

13. The expression vector according to claim 11, into which a desired gene is further incorporated.

14. The expression vector according to claim 12, into which a desired gene is further incorporated.

15. A transformant comprising the expression vector according to claim 5.

16. A transgenic plant comprising the recombinant vector according to claim 5.

17. A method for producing a stress-resistant plant, which comprises culturing or cultivating the transgenic plant according to claim 8.


SEQUENCE LISTING (20 sequences)

SEQ ID NO: 1 - DNA, 2072 bp, Arabidopsis thaliana
tgggcttttgatccacatgacgatcatatagttctttccttcaacacaaaacacttgaagtgtatcttatcaactaaactctattccagcaaaattatttcaaaattagatatatgggagaatttttacaggccattctagattttcattaacgctgtgatttatccgttaataagtacgattaagagtaaaacgacgtcgtttgcaattgaaacaaaaatccccaattcagaaataagaaaccctagatcctcaaatctcttcttcttttcttcttcgaattcactcctgtattttgaaatcaaagtgaattgcaaggcacaaagttcatgctttaaagcactgcaaatggcgaagtcatgatttatatggcatttttcctgtttttgtttcaattgcaaacgacgtcgttttgctcttaaacggtctcattaacggataaatcacatcttctaattgctctgtttattcagttgatgttgtgactaatttggctctgtcaacgaaagttagtgattttatctgaagtcaacacgtaattgatgattttaatcacaaagtacaaagtttatgtttaaagcaccacaaattgcgaagtcgtgatttaaatgacatttttcccgttctggttaaactaagtttctattatttaatatggatattgtggagcgtagtgtcagcactcagcagcttatttaacgtgatactctttaaccgagtaacaactagtgaggtcatattcaggaccgatccaacaatattgagggttttactccaagtaaaattttagttttatttttaattatcataaacgacataaatataatatggaaagatcacaaatactgattaaaaactaaaatcatcaaaacgaaaaagaaaaaagaaaaaattgggttcaactctcatgagttattaaacattttaggttttaggcttaaatctttaaaaaaaatcagaactgaaaaacgaaaaattctaattttattttggactctgattcatagcttatgtcgcttatgtagttatgctagggatgaatctgtatttcgttaccgtaatgagagttcgatactctcttacttgttacgattctggagcatgttacatttttttctttccgtcaacaacaactttaatatggtaaaacaaaatttatttttatttggctggtcctactcaagacaaatcttctgccgacatcacataatcatattaaaaaccataacttctgccactctgtttttttttttttttgtaaccattaactgattggattttgatccatctcatctgattttttagctcaacaatttacttgcacattttctatttggttttatttatacttagttacatatatgattatcgaactagtatctctttataattaagtatttttctatttttttttaatttagatttttgtgaattcatttacagtagaaaactgtaaaaccatatggtctaattatagaatgaaaacttcaacgaatccatacaacttattggctaaatataataaatctgcttgaagcatattgttattatttagttggatttgacgatctctgactttaatgtataccgacataccctatgatttagatgttgatttttcccattcttaatatatccatgttaagagattccaccataacatatctaattatttgcattgtaataaatattatcattaaaaaaaaatacaactggacagctggctcgtcccattgtttcttacgtccaccaattacatttgttaaagcaaacttattagaacgttcatgtgtgagaagttggtgtcgacatgtgtctaaggtctatgtcagaaatcggattagcttattaagtaaactatactatatcattgttaatatagataaaatatctagttcgtccaaattaaactattttcataactgccacgtggcgtaaacgtatccatcgagtcacttgtaatatctttataaccaaagtcttccaacacattcatcaccatctatctactctttactctcttctcttctcacatcaattattcatagttctctcttctccggcaagaaaag

SEQ ID NO: 2 - DNA, 2000 bp, Arabidopsis thaliana
ctcctgtttcatgatcatatataaaaggaaagagattgattcagctaatcaaattcataaacatggataagatatcacaaactctaaacctagctcaaactatgaacaatagaggataaccacttgaaacctaattttaatcagaagattttagtacttgtgctgcttattcaaaattcctttgcccaaaaaaaaaaagtttgacttaccttttgaaagacgaacgtatccactatggaacaatctttcgtaatctcactttctagtttggtatttaaaaaacccaaaatttgattgtcaaattaaatacaataagagattcctaaacccagaattcaattgcgaaatcaaacagagacgatacagagagagcgagagagcgagagagagagagtgatttacgtgagaacgaatcggataaacggaaatttccagaagccggatgagtttcccgctattgtgatgagtaacaaaacaaattcgggagttgtctatttaatacttatcaatagcccatgaacgcagagacgtatggacgcgagaatcgttagtacgtgacacgtgtaaaccagtgacgtcaccgctctcgtcacaaacccgctagtattgtcctcttctattttatctcacacaatcatgttaggttatcatagtaccactaaacatttatgtgagaatggatggatattagacaaccgacgacaaataaaacaaacaaaagtagatagacgaccataaattaaactataaacgaagattgagggaaaaaatcattggtttagattttcgaacaaatatcgtgaaaagtttttcatcttgtagtttttgtaattaatggtgtatgaaaaatcattggtttgtttagttgaattaaatttcagtaaacggtttagagtatgaacccactcaataaagatttgactgccaagcgttactggtgtctggtggattgaatagttaagagtataaggtttgggccattatgtggtttttattaaagttgaaggtctcatttattaattccttagagctgatggctaatgtacaacgaatttgattcaggagtaagataaacggtacacccaaacacgcgacgcgagcgaagaagacgatgacgaacaaggagaaagctcgagagagaagggagaaaagaatgcaggagatctctctccttcgaactattccttactctgaccaccataggtatgaagagacctaatttgtccaattgtttcggcttctttgtatcctaagatctatcatcatttactctgcgagattctcaatttcacagcattcgatgtgttaatctctaatttcacttgcttaaatccatttcttagtgtcgcttacagtgtttgatgagtttctatttttctctatttgggagttggagactgaattggtattgttgttggtacttacagatttgtgtttcactaaaccaaaagaatttagagaaattgaaaatgatgaagtatgtgtctctcaagccctctttttgaatttgacttgtcattaggcgtcaaatgattttttaggtggtggtcttgtgaaaatgtagcagtagtgactggttcaaaccgcgggattggattcgagattgcaagacagcttgcggttcacggattgacggttgttcttacagctagaaacgtgaatgctggtcttgaagcagttaaatctttgaggcaccaagaagaaggtctcaaggtttattttcatcaacttgatgtcacagactcttcgtcgattagagagtttggttgctggcttaagcaaacatttggaggtttagatattctcgtaagatccttatcgattcgtaatcttggaggatagttctaatgttttcttagatttgaattacatgcatttgtgttgctatgtttttcaggtgaataatgcaggtgttaactacaatctcggctcagataatacggttgaatttgctgaaacagttatatctactaactaccaaggaaccaaaaacatgacaa

SEQ ID NO: 3 - DNA, 2000 bp, Arabidopsis thaliana
catataccaaccgcctaacattacagcctgtccccatcccctatctttatttttctttttggctcatttagcttcaactttttttaataggtgtgattagaacatactaaaaaaaacatatgttttaacttatattatatgaataaagtgttgtttttaatagtttatatttctattacaagtacaatatataggatattttggttaacaattttaaatttaaaagatattgtgagtacaaatcattaagcttaacacaaatgttcattgcttcttaattcttacaatcggaaaatttggacaaaaaatctagactaatctccaaaacattgtgaaaaattgggggttttttttatcatttaaattgtctatagtggtcaatcattattaaaaaaaattgttttttgttgaaattttaaattataaaaaaaaaacacgtttatacataaaatgcaaaaacttttcttaaaccaaattacagagaaaggctatcaccatccttgtcttgatgccaaccacacttgtaatcccaaaatacatacttaaactatactatactaacatgaattggttatacgattatctatctagttagctacataattcatataattagttttttaaaatatacttatgtaatgatagtaactaaaaaatatactatctcgagaaatccaaccaaaatgctatgatagtatataaaataaaatgaaagaaagaggaaccaagttgtgaaaccatttaccaagaagaagaagaaaaaaacaagatggtcgagtctcgtgatagtatgaagctggggccgaaaccacgaatcaaagaaccatttgacgatgcaagtggtcatcgataaccaatcaatagaaactccaccgcctcaaaatatcatagtgctatttaatttttcatactatcatttattgtaggctataacatatttttgtattgtagatggacggctagtacacgcaagttagtcaataaacttttaacaaatggcatcttgacatgcttaaacacattttataattcgaacctcactttgcctactttacacacgtctacttatagtagatgagcatcaatcattgaaccgttcgtataatacaattttttttttgtaatacgttataattagaaagtaatggaaaataagttgaaacacaaattttcaacggttttactaagatttaattttacaattttcaaaatataaccgaattatttaaatcgtattaaatttataattaattttctaaaaaaaaacacagattaaaaaaatatattaaaactaatcataaatatattaatttttgtaatttatatttttttataactttaaactaataatattcaattatttatcttaaagtttacaaattatcaataataactaataaataatgcataaaaactttttaaaaattaactttttgaaacaaatttttttctaaaaaattaaataataaagaacagaaaaaatattattcaactaaaaatattggataataactctgaaagtttgcatttttcaaataaatcattgattttttttgtttgacataaaacattaatttccccttgtaaatcacacatttaaaatctaactatatttaaacaaaaaaaaaaagtccaattttgactttaattatataaaaaaagcaaagctaattatgggtcaaaagactcaaaggccagaattgacgcagccgtttatgagaagtgagaagacaatttcggagtcacttcctttatttcctctcttccttcactctctcctatataaaccttcctctcctcttcctctcttctcatctcttcaaaccattttcgaaagccttgagagagagaacacagacgataccaactttcttcaatctcgttgccgcagtataattatctcattcctcggatatatctctccttctgcggcggcgacaagaagctacaagaataaaaagtctgttttctctcttttcaagaaaccacttacttcgaaa

SEQ ID NO: 4 - DNA, 2000 bp, Arabidopsis thaliana
agcaagtaggttacgtggaaggaagatatcaacgcacaaaaagagaaacactcaacgttaagggaccaaccacaaatcttggatttttgctgatgcagtgtgtgaaaacaggtccacacgctttgtacttttttcatcatcggtctacaataaagctcaagtctttccatggacagagataagcaagaaaaagtattaacattgttcacctcaaaataatttaggaaaatgacaatgagatttttacctcaaaagccaaaatcaacatctacccattctttcactcgtcacaatcatgagtttcttacaatgcttaccttagagtttagagacatattctcacaaaaaaagcttgtgtctatttcaaattcttgaccgtagatgtcacaacatgcatatatcattgaaaacagagcaacacaggaaaccaagcatatgtatctagatatacttagcaagacataactatagtctttgaatcaacatagggattaatgatagagaatgaggaagctcaagattttatactcagtttcttacaaaacaaatttctctctaactgcaaaaacaccaattaggatttgaagagcgtacctgtttgagtcaatgtccaatgtcgtccccccgccttctacatttcttagcctgctgaataaaagcacaagccaaaatgagaaggtgccaaaggcgataaggatcaatttctaccattcaaaaaactaatggtgagaattagaaacgagagaaaactacttgttgaggaaatagccaaaagcgcaatcttcgtcacctgaataaagaccaaaccgtcactttcaatgagtcagcaagaaaaagagagagagagagagagagattctctataacatttgagtcgacatggattctaatgcatcaaaagtcatctccaataaacaaacacttgaaactcacatggctaatagaacaagatcaaagccttaagtattaagcattacagacactactggctaacttttgacacatgttcttaagtaacatagtatcaatatccgtgaatcacatcgaacacacacaacaagggcttaatgcatcaaagtcctgttatttccatataacaacatatttcatttacaaacagaatgcagcattcaggcagtccaaatggaaaggttgacaaaaaaatataatcttgtaactctacatatatggcagaatgtaataaccaggcaagaaaaaaacagaataaacagatcaatgagtatgatataaaaaaaagtcacaaagaatgtgccacagtgaacaagagggccatgagaagaaattttcaaagaaaatattagcattgttagaattttttgggtcaatggatctgtcagctgcttagttggaaaacacaaatcttacaggaaggaaagtccaagaaaaagaaaataagcaaagttaatagccaccacaagaaatttcatacagaaataattaaatcgttgcacttatcttcttattcaaactaaaatcaagagaacttaataattttcagccacacgaaccatgtgttcaaagccaaaggtgagaagccaaaattatcagcttatctccattaacaagggaaaagcaagactagatttaagagttctctgtaactaaaaactgcaggagtgagtaagtaaataattcaccaacaggaaaacaaaactcaattatctatagctgaatacacatgtaaatgagaatttattaactaaaacatcttcctttgtaactgatgtgacatttacaatttttcattttgaggtgtaagaaccgtgtgacaagtgaaaaggttaaaataagcaacctttgtgatattttctctccactttttgaaattgggtctccaaaccacagccaatcaatattctttataaatacaaacacacaaacagcatctttctctcaaacacaaacatatcttctatcaaacaccaacagctctattctctacctcatttctcatcataacaaagagagagaaaaaaact

SEQ ID NO: 5 - DNA, 2000 bp, Arabidopsis thaliana
tttctggatcacgttcacatgccgataattattgttaagttgtttgcatattttattatgtgccttgtttgtgggattttcgctaatttcttttctcgttttttcgttctcctcttccctcaattattttctcaacttcctattcgaccaaagcattaattctacacacgacagtaaaggacccactcaaacaataattagttttcaactctttcagaatatattttagtgacactaaaatagcatatcataaacacaacagaggccaacgactctgaatgatccgtaccgcagtggaaataacagtccggttatgtcggtaggctatataagccactactttcatttcaatttctcaactttgttgtacttagttcgtatcctatcagtttatcatcactttaatttgttaccatatagtatctgtaaaaactaattaatatttatgaatcatgctcatattttcaaatcttagagctactcaacagaacacacacacatacagtcaaaattttatactacataatttttgttttaagtttacagttataaaccaaacaaatcacctcacaaaagtatatagttataccaaaccaagcacctcataaatgaaaacaaaattaaaaataataatatatataataagaaatattcatcaatactaacgttcaattttagtaaattggcaaaaataaaatattagtatcacttccagagattacattattatttggtgaatttggcaaaaataaaatatttcgttggtggatgctttgctgggacgactttgtttattaccgtcatattgtagtaacctaattttattacctaacattcatttgaacattaaaaacattaatgtcatttgattggaagagtttattaatgaccattatttccttatttccaaacctaataaacccattatttccttatattaaattatggcttgattggtgcaatttctttctagctctttctgtttccgtcttgattcgaataaaaacgacaaatgttatctctacccgtatccgattatgtttaatatattatgcattgatatttcacgctacgatctggaccgacgtttagtgtggaccctgtcacgagttttatctatgacggaccaaaagaaaaactaattaaagcagaatatcaaagttagtcgggtaagttccaactaataaccgagccctgtttggctttgagcattagtagtaatcaaagtaaacatatgatagaaaagttattataatctattatatacagattaatatgtacgattccagattctatttatgtttctcgtatgtaaaacatataatgtgaacctacttaaactatactaagactattagtttggtttcaaattagattaattagtttaaccaaactgaacatatccctagattgtaaaagtctaatatatccactaattatttttgcttgccgacctctaatccacaactttttgtttttccatttccactgattttcaacattccaccgggtgttaccaacagtaacatacatacagtgaattttttcaacactccaccgcatgttaccaatagtagcaaaaccctcgacaaaaatttgtaccgactataaatattcaactccaactctttcctctccacacactcaaatatctctccaacactaaaataattgtaccgactctctctctctctctctccgtaacaaaaaaatcaccaatggcaaaacaatatctctttgtactcctctcaatctcctatctcttatcactggagctcacggcggccaccgcagcctcacagaccggagcttccaaaaaagccataaacttcatccaatcttcttgcaaaaccaccacataccctgccttatgtgtccactcactctccgtctacgcaaacgacatccaaacaagccctaaacgtttagctgagaccgctatagccgtgacactaagccgagcccaatccacgaagctcttcgtctcgcgtctaacacgt

SEQ ID NO: 6 - DNA, 2000 bp, Arabidopsis thaliana
attatatctataataacatgattctatttcaatcgagtatgtaaaaaaagtaaaaatttgatcgattaatcatgtagtaatctcgtgtttttttccaacaaataattgattaaaagtttaaaacatatcataaatcaaaaccgaatgagtaatccggattgagtctagaattacaagttaagttatctgtcatacggtggtaatctcatattttttgtagaatccttaagagttgagaactttcaagaacagttaagtttaataatattttaatctttgtataagagatttcttcattcttctaaaaccacaacaccttgtgatcgacaattcatacgtacatatttgagatttaatttgttttccttatataagaaatcaataatcttattgtatatgactaatttattatataaaaacccttactaggggttatataagacacgtagaaatgtctccaacatacagtactataccacaaaattcagaaatttgaacaaccaaagcaatcagaaaacaatattcatttttgttttattggaaaagattgagccaatatttcttaaataactcctttttagataatcttggaaccgggcttgttcgagcgggtcgtgccagccgggctgctagggctgttggtagccggactcatcccgagagtgttcttcacagcatcggctgctccttgtgccatgttcttcacttgtccacccgtctacactccaaaatcataattaatgcatataattgtctataattaataataatgaacaaaatttttgagaagaatcgttaacctgttggatgacactagaggcttgagaaattagggaaggattgttctgatcatgctctgagtgtaactgtgactgtgagtgggtgtgatgatcagcattctggttcattgcgtgtgatacattgttcaagtactcttccttcttcagctataaataaacatatatttgatggttcattcatcacaaaaatcagatttcgaaaattgtcaaatttcagatcatataagcaaaacaaatgtcgctattttgtgtgttgtatgtacctgaacttgaccggtgacttctccggcgctgtggctcaactgctggctcgacgacatcttcttggttgttctgtgaaatgtcttaattaattaagattgtaagcttgtagaatacttgaagggatttgatatgcattataatggtatttatataattagaaagtgtatattttagtaaaatcctaaatctaagcattacactaacacgtggaaaataacataccattgacgattgacatggctaattttttgtggaggtgaatagtttgaggatttattaccctaacgttgcttggtcaagaagtgaagtaggatgacaggcaataggaagatcttaaacctttttttccggtgacaattatttatgactttttattgttgtcaaaaaatatattatcagtaatatatcaataacgaatacaataaaaactcatccgatcgattttcaagaatttatagctatattaaaattacttcgaatccatgtaagaattgtgtattggttctttttagaaaaaagtaaatatctatgcagtaatggcggttgcataatatatgccttgagtagatgaatatccaatatcaagataacgtgagtcaccacgtgtctaacatcttccgtagctccgtttttaccatgacgtgtcacatagatataggtcatcatgaaaacgagaaacctaactttaacactcgcacataactccaagtttcgaaacttcgtcacatcaacctaatcggggcacgtacctacacacctgtcgcgaaactgcaacacctatcttgttctctcgccgaccaagacttgctataaataactctgactaacgagtcggagacaactcacagttccaaacacacaaaaaacacaagatctaaaaaaaaaagcttttatcatttagaaaaatttggtttcgaatttcttcgaagagtgaaa

SEQ ID NO: 7 - DNA, 2000 bp, Arabidopsis thaliana
tgaacaaatttaatgcagaatttagtggaggatcagttgtgattttccttcggtaaaatcattttcagttattgacatttgaattgttcaatttcaaatttcaaatattataattcaattcaaagactagctaagtttttaaaagtaattagtgagagatctcacagtgatataaaccacattcattttaattcatagcaaagtaaatggtaagaaatgaaaatggaagaaagagaggagagtagttcacacgaacatctgaaggagaagttgagagagcttgaggaggaatggacagccatgaaaacaggtaaaaactcatctgcggtttcatggattaccgtggaagatgctcttgagtatgtcgaaaactctcccagaaacctaatgctctctctccaacacaaaccaaaggcagagatgatacaagaaatatcgccattaaggagaaaactctttcacgactctgatgatgatgatcaaactaagaagacgacattgctttctcattcatcttgttggtcttcgaatgtgacgagttcgagtgataccacgaaagcaaagaagaagacaacgattagaagatttgtttctgttacaatggtgctgcttttgtcatgggttttagttgtgttgatgaaccactttgatcatctctccatgaatacacaaatcattactttggttcctacttgatatttttatttattttggtttgagaatgttcatcaatctcaataaataaaaaaagtttagttttgtattattacctctcgccttctcttttaattcattgaacctcttcaactttggctgtaactatgttggttaaaacagtataagtaatgtatgtatttatggttttaggaaaatcttacaaaataatactctatcattggatgaagaagcaaaaactacacccctctacatcttttctattcataaacttttttggggaaagaactacattggtggtgattaggtgcatcggtgacactaatgttgaaaatatgttttgtgtaaaattagtatggttaaagctaatcatttagttgagcaaaaaaagatgctaatcatttaaatagaaaatggactcaaacaaaagaaggccacacattcaattgttacatctttattgggttttcatttttggaagctatttttatgaatttgcagtgttttgttgcccaaaagaaaaaaagcataattccaagtatatctttcattttgaatccatcagtcatatgttttacttgaatcaactatttgttgggttttacgaagttacttgttaagaagattcaaaatatatataattagactatcaaaggaacaaaagaatgtaattgggtcataactttttccgaagcttacgtgacttgggagaaaatcaaacccattagtttcacccttaatcaaactaatcacaaattttgaactttgcaaagaaggtaaaataataatcagcaaaaagattaataaaatgacaaaagtatctctttaaaagattaataaagaaataaagaaatgacataagctctcaccgaccgacgtcttgtcttttttattcttaaggtcggtgccatgtcgacgccttcaagcttagaatctcgccacgtgtgggataaactaaccgaacccaatcgaaccagacagaccaaagacagaccagaccggactagttgtatgtcggcatcagcgttctagtacgacgttaattaaaaaaataaaaataaaatattatttatgatattccaccaataagagccaatgaaaagtgtgtgaaccaaccaacttctgtagcaaaccgtaccatttctctcttagatacctttaccttccttctataaattaggttcttcctttcacatttctttgtcttcttcttctacataaaattgattcgcctcgaaatttacacagacttgtcgctctctctttctctcttatttcacagagcccctaaacatttccaaaaatcttgcaaaggaagaaaaaca

SEQ ID NO: 8 - DNA, 2000 bp, Arabidopsis thaliana
aaatatcatcatttatatcaaatttatgatatataaaatatcattatcattttaaaataacactaaaataatcagtgcaattccgtataatcttcatatgaaactgcaagaatccttggtgttccaaagattatgatgattattgtgagctttgatttatggtttccatcttgtcaaaaaatggcttttataaatggtagagctttcttagttttccttaagatatacacaacaggttacaaatataatttatttttgctaaaagaaatagttatagtaaacttgtaaaaactactacaactattttatggaaaatgtcttaaagctgtttcaacttttatacacattactgcatttactattctgaaatcaatttttaattaaggtccttgtactataaggaatcctttaaataaatataaatattaacaataaaaaaatccaaaatttcagaatgcaatggtcttacaattacaataataatgtatttgattgtactaaaaatataccttaaatactcaccaattatcttttataatataatccacaaatttctggaagaggaaaaacaagtaaatgcaagtcacatttcacatttcaaattaccaactaccaactacaaactacttccgcgtctcaaattgaaccactaattaatcactactttatatgctcatcattctttttctttcattacaattctagaataatgacttaaccaaaattcatacaaataaaacaatattttggcttccttcaccaggctaagaattttggtattgaaaatctaagtacttcataagaaactgttggaatcatttaaaatttgtgatcatatataaaaaaatttattcatctttatatttaagagtttaaaaactgcaacttttgtttttctttcactaagtcttatggccacagttaattaaaagcagatgaaaggtggtccaatggaaaaggagaatgtgattgggctagttgggagagttctgatgtctagtgttgggtacacgtgtccgtcagttacacatagcattaaatcagacggcatgtcattattcaaatctagttcacatagtacgactaatagctgataaattaatgattatacagcatatgaattatgaattcaaaaaaaaaaaaaaattgaaaatgttaaggagatgctatattttacaaaattcatcgcaatgctttctactaatttgctaagtggtcttctccagttagtcttgtcgattccaagcgatattattaaatcttgaagcatcgctcaaagcattatagcttaagataaccaaattgttattaaaaacacctagtgaaatttttaaattaaaacaattttgatatctttgtaatatctaatactactctttctgtgtctaaaaggattaattttcaaaaatttcacacatattaaaaaaaaaaaaaaattactagctaaacaattttcaataatcataaaacaatagtaacttaataatttttttttattttcaaaatagtccttcaagtttacaattcattttagtattataatcaacaaaatttgtattaaaaagttggaaaattaatctttgtggaacaaaaaaatctagaaatcattttttagaattagagagaggtttgataaaaaaaaataaaaaaaaatagagagaggtagtacatactaaacgatgtgatactactattgacaaaatcttaattctcagtttagtagaataaactagaaggaatgaatgaagtaaatgcgaatccaactactaacaaaccctacttagtcatcatattttcccatatgaaatccctatataaacccatcatcatctcccacttttttcatatccaacaacaaaaacataagctaagaaaacgaaactcaactaattttgttatcacccaaaaagaagttcaaacacaatggctttcgctttgaggttcttcacatgccttgttttaacggtgtgcatagttgcatcagtcgatgctgcaatctc

SEQ ID NO: 9 - DNA, 2000 bp, Arabidopsis thaliana
tcaatttgagacgcggaagtagtttgtagttggtagttggtaatttgaaatgtgaaatgtgacttgcatttacttgtttttcctcttccagaaatttgtggattatattataaaagataattggtgagtatttaaggtatatttttagtacaatcaaatacattattattgtaattgtaagaccattgcattctgaaattttggatttttttattgttaatatttatatttatttaaaggattccttatagtacaaggaccttaattaaaaattgatttcagaatagtaaatgcagtaatgtgtataaaagttgaaacagctttaagacattttccataaaatagttgtagtagtttttacaagtttactataactatttcttttagcaaaaataaattatatttgtaacctgttgtgtatatcttaaggaaaactaagaaagctctaccatttataaaagccattttttgacaagatggaaaccataaatcaaagctcacaataatcatcataatctttggaacaccaaggattcttgcagtttcatatgaagattatacggaattgcactgattattttagtgttattttaaaatgataatgatattttatatatcataaatttgatataaatgatgatattttacagaatgaaatgttgttatacatactataccttaagagcatataacatgatataattgctacattagtgtcggtagaggtttgtgtagtcaaattgtgactattaagtactacacatgtgagacatttatcaatcttgtacgatgttgcttgacggtttttggctgatagtatccaacaccatacatcaacctttcacgtcccaagctctctaagacatcggagaagcatcatcacatgcgtacgtagaaaggaaacacaaaagttaaagacattttgtaatatgtgtaactatgagacatatatgagactgcatgtgactatgagttatatatggcttatagagcgatagtgttatggagattatattgcattcatgctatgtactgcataagacgaattcggtttcgtgtatttattcccgcttgagtctagaattggtttcaagtatagttagaaaaagactgtttttttcgtcctattatggttcgtatacaaaattatgcattgaattgatgtggcattcacgtaaaaatcttcctgcagggctataaaacgtatttttggcgtacgattataataaattttgttattattcagattatttattaaatcttttttgttgttgacaaacgcatttatgttaatgattatacgaaatatcgagaaaaactccatatagcacctctatcattcaattatgtagtaagtcataaaatgtgaactttgctaattataatggccataattagtaaacgtgatgggagaattgtgatgtttagtgttggatacacgtgtctatcagctgcacatcgcatttaattggacggtattacatatatagtacacataatagctgacatttcatctacaacgaagatacaaatttttaaacgattttctcttgtctactacagtataacatcgctctaaacatgctaattaaaaaaacaagtttaattaactgcagataaatgaatatatattttttaacagtatagctgtatatatattgacgtattggtctttgtacattctgaaaactgatactattgtttacataattacatatgactggagaaactaattattttggttagtagaagaaacataactacatatgactggagaaactaattattttcgttagtagaagaaactagaaagaatgcatgaagtaaatgcaaaaccaactactaacaaaccctacttagtcatcatttttcccccatacgaaatccctatataaacccatcatcatctcccacttttctcacatccaacaacaaaacacaagcgtagaaaacaaaactcaactaattgtgttatcacccaaaagagaagagcaaacaca

SEQ ID NO: 10 - DNA, 2000 bp, Arabidopsis thaliana
ttttcattgattacatttacaaatttttggttcatgttattagtaaaatagatgtttatgaacaaaattgagctaacatatgacctctcataatatattgttgatatcagttttaatatcaatacattaataataccgggaaaaaaaatcattcccaagttttgtaaagaagatgttcatataatttaagtttaaaagggtataaccaacaaaatgattacaaatgcgtcgatgggataaataatagatatgagagaaccatacaaatatgatagtgaacaaaataaaatctagtataattatgggtcaaaaaaacataaaaagaggggaaaagaaaagataaagggaaaatcaaaagggatgaggcaaaatagagagtttcaataaagataaatttgggtgatggtgacggaataattcttcgatgttttcgttttgaccatcttttcctttatttctcactttaattttcctttatttctctcgctgtttattcccataaaatttttaaaattaactagaacaaagcacaaaaatgggcaatcttcgaaatatactaaaattttctattataattaacaaaataaaaaagagagggaaactgctaatatctggagtccattctcttggagaagagtaaagtgggcaaacaaaaaaaaaggaactgagctgattcgattctctttgggactcccatccatgcatcacctctttcatagtttcattttacttttatgtttattattttacaagattaagtaattagcttttttaactgcaagcataacaaataacaaaacctttattacggtctcaaacgctcaaaaatgtttttaaacatagtaaaaggtttcgttcaatacaatttgaaaaaggaaaaaacaaaaacaattttcttacaaatagatggccaccaacgattaattattaccgtcaaacttataattgggatgactcggtggttttgcgatggcgtttgttccaaaagagtgccatattgttcatttgttcttatatatgttttaatccataacacgttttggatcaatgttttcaagtaatcaaagtgaaagtagagaatatatcaattggtgaaaattttgaaatatcaatgtaattctttttaagtggatccattgatcaaggaggccactaaaatttgctcgaggcttggtagtgggttcatttgtgtttgataaatacactttttagtactcatcccctttttttgtctgacatcgtatatgtatattggttacatctaaatttgtcacacttgtgtagcagtgatagttgtctcagtgtgtctatagactatagttaaatgttaattagtagtatttatacaatactaattacatcatctaaatatttgcatatgattcacacaaggcaaatttccttggctaattgtcacactatttatgtgaaggtgagtagttaaagctaataacctcgaagttgcttggtttatatgagatcaagtttggctcttgtaagtgaagataataatttaaagagtattttaatcgtgataatctaaatgttagttgatagttaatacgtcacggaacatgtaccgaccgacggccgatttccatgatacaataaaaaaatgaaaattagcaatctcataacatgcgacgacgatacttatggtgttgccacgtagcaagcatcttcctcttaaccatgaggtgtcgcaacctcagaggacaacacggacaagaccgagaaaccgcatactaacgcttgcaacgtaaagcacaacacccctaaactcctatttttttaattttcttttaacctcaacacaccacgcagctatacacgtttcaatgtgctaatacgtgtcttcttctccgccgaccaagacacactacaaatgtcccgatggtttggggagacaaatcacagtttctactacaacaacaaatacttttacgaaaaaagcttttaaaacttatctagtttcgtttgatacacatttgaagaaagaa

SEQ ID NO: 11 - DNA, 2000 bp, Arabidopsis thaliana
attaccataatcataatatcatggtgccgactatatggctcgagtctttatcgtttatttttatttcttaaatcattgacttataaaccgcccatgagcattaaataatttgtctcaaaaactcgaaagaaaatcgttattgaaaatggtttaataaccaaatctaatcctttccgactcatattggtttcattataacacttaacaagtaaaagaattttactcgactgcttcagccacaatgcggtagaagtcgccaaaggaccattcttgatcgtcatttttttcttctaagcgtttgcggtagcaattttcagccatagcttccaagtctttcgcgtctacctctcctttaccttttgcttcgttccataattaagtataccaaacaaaaaaatgataattttattaatgaagtgaacatcaatgaaagacgtgattttgggggagaaagagaaaggacgaaccggtattgagagcttgacccatattgtgataacttccttcttcttgtttttttgcatatcgtaacaaatttgaaggataatattgagaaataaattttggagaccgtgacttgtgacttgtgaggtgggatcagtatcttggcatttcctgatcgataagccacgtaccggaagcgtgacagtttaacttaaggatctagagaaaaagtgtgagtctattcatttcatattttacatatagcatataaatactattttattttcctttgtttacagtttaatagttttagactcacaatatgacttttgaaagccaaaatgtagcagaacaattaagtttttttaatattccttttgaaatgcaaatatgtttttaacctatataactggcaacattaaaaggaagagattctcatgatttgcaaaatatcaaggtggaaaaggcatgtaaatctttatttaatatatatctttgtttaaaaccacattggagataattgaccaaaaaaaatacaattttgtcaaaaagcttaaattattggattgttaatgagcatatggaagtttgacacttggagaaaattcacatgtcaccacatagagataatgtatttctatgttcactaacacgattacatttacaccccattccctcgtcgaccttgagtcactcccatctccttctttggactcttgcgagacacttagatttagaacaacaaatgagtttaagtaagtactaagcctatgaaaaaaaaaaaaggtagaataagcttaagaatccagcaacattcatggtatcattcgccatttatatcttaaacgtgaaagaaataaagagagaaaggttatgaattccttttgtcaatagatatcattggaactaagcgttatgaattctttttgtcaataggctaaaaaccgttttatatattttacaagaaacatgatttttccataatattgaaactttatatatttcttaatattctaggcatttataaattattctatgttttgaatatttattttaaatttttggagaatactagaacatataactttccaagtcctagtgatcccggctacggcaacacatccctacgagggatcaatatatcgaaccatctcaacgcaacacttgtcgtcgatcaaaagtctaaaaacattgcttctcaactttgatctctttcctctagtctctatcatcaccgccacgtcactattttcccgccttataactaggggcaatcaactctcgcgaacagagagagacggagaacaagagcaatcaaatcacacaaatcctcgaaaaagtaaaattcttgattaaaaaaaaacggacattttgtgctcagacactcagctttccttattagtcttttgccacctctctgcaaagttctcgaacccactctgtaaccaaaatcaatcataaagatttgatctttttttttcctttttcggtgagatttaatcctctaaggttcttgatactctctagatctattcttacttgggtttgttcgaaat

SEQ ID NO: 12 - DNA, 2000 bp, Arabidopsis thaliana
aaattaaaaaaaaaaaaacattgatacaattcttaccttcagtttttaaaatttcctgtgcactatttccttccttgggtgtcttgcgactgctttttgactttgtttttggagatttttcctttcgaactgtcttaactgctggttcttcctctgtagcttcaagaacaaaatccgcatccttttcagttgacacagtcacttcctgctttataggagaggatggctcagaatcagatgagacaagccatacagaatcagctcctccctgctaatgatacatttaaataatgttaacatatcttctcaacactataccaaattaagagttagtagttttacacccctacgatattatattccataaattaaactgatccatgcagacacaaaaccttgaaataaaaaactaagacagataagagtgctggcgatagatcaccaactaatttcatcatatctcatagaagtccaaagctgtcctagagttagtttataactagtataccactatcttgacaatacaatccatttatcaggcagatacattctgacaagaaacacttgaatcacaatatcgacccactaaagcaagtagaggcaacagaaacactaaggagtacgatggagacagtgtccaaaaggttcttacttgagcatccttatgcttgctagagagtttttcacagtcaacgttgtttccattctccttccctgcacacgaatctgcccgagtcaaatacatgcttttaagagctcaacagttgaacaatactcccctaaattcataaattgatattctctccctcgccctccaagaaggaaatgacccataaatgtttgttcctctaatctcaaagcaaaacaacacagaaaatcttccataccttcaaggttgagactagcatctgctttcttctttctagaaaacacctgctcgatactcacttgcttcgtcacaaccttcgtttcggaattcttcctagtcagtaactccacagattctgtctcaagaaccacaatgtcgtcaccgtcatcatcaggcaaaggaagagacgaaatgacttcagattccctataaggactatcatcatctgaagatgatagcgacaacaatgaagtagtcatgggtgcctgcgtaacaaaaccatccactttcaattgttaatttctatatatttcaaacaaatatataaaatagaaacagaaatcagattgaatctggaacctcgtaagagcgaagccaatctggagatccctctctagagctgctgctcataatttcacttcttcagagctcaaaacaacaatctaagcttcttcccagcttcaatatccactcaattacaacaaaatcaggtcgtacattcaggatttacttcgcaatatcaaaaccctaaaaagagatcttaaaagctataaattcgattgaaaatcacatgaaatcgaagctcacatgaagaagaagaagacaaaattgagtgattgacgaatctaatgaggttaaaacccggcgaggagacgtggaaaaggcatctgaattgactgaggcctgagggaagaagcaatgatttcgccggcggagagattgaagtgtgtgaaagacgcgggaagaagattggggcataaactgcttgttcgagtttccaagtgtttggtcacaagagcaatactatccccaacgtcgacgtaaaatttactaaaagcccaatatgttttaatagaaaaattaacataaagcccaaattcgatgtaatgggctttcatctttcaggaaaaattaacattagcccagatatgccaatagagtagagataatgacgagtgacgacataaaaaaaggcacgcaaagaatataatattaatttcttggtggacaagcatgtttgaccattctcattttcttgtgcccaaacttatatttggtatgctcctcctacatatagacaagacaatggtaattcgacaattcatcaagtctcaaatctaaataagtaaac

SEQ ID NO: 13 - DNA, 2000 bp, Arabidopsis thaliana
agaaacggcagtcccacgagtatctgtataaccattcattgaccagctgaatggtacttcttgcataatcaaaactataattcgctggataatgcaactcacctatttacattgaagaaactacagttaacaagtttccaaattgtcttggagaaacaatgctacgcatacaaattggtgaataagaaggcttataagagatatagtgaaacctgaacaagagctgcaccagccaaagcagacaccactccaatgatattcatggcctgtgaatagtaaaacttcacatatgaagaaaaacaatgctagaagaataataaagcaaatccataaactaattctcttccacgaacatatataccttaagtagcagaagtaacaaagttggagcatacaaaagtacattcatctttacagaaacagctccactgtataaaaagaagggtaagcagactagtcaatctaaagaatcgagaagcaaaagcagcaatctaacctgaaaacaagcatccctagatgccatttacgatagagaaagagagccattgatgcatgtaggagagtcatggcaaaacaatcattaaagagacggagaacaaagattgaatgtatcctcttggataaacatagtagacttaatgcccaccatggcacctgaaatacgtaaggatacaagaagagatatcatcactgaattcactaaaatcactcccatcataaacagacaaatcaaaaccaaaatattgttcattaacatcgtaaatggacaacgcttgacaacgtctacaaatacttgatgaaaagagataagaaataagtcttcatttataccacatcagtcttcacgtagatgatcaagacgatccccagattgacaatgtagagaacaccaaaaagaatctgcaaaatgtaagagagtagaattgagacaaatgaagcgatggatggatcaaattccgcagctaatccaaagataggagattcaaagagagggaattgcaagcacctgagcagggtagacttcccctccagttaagttctgcactgcagagtaaacatagaggaaacctgctggataaaccaatggccctgtatcgcctttcaagttcccatagtctctctctcccccgagaaacccactaacctgccccaattagataaatttcacttaaagactactagtttactttgaagaaaccacagttgaccattgacacttacctgtgacatgtaagcatcccaatcgatcttcgtgtctgttaaaggcaaatgacgaaatttggtttaaaacctatgcaaatgcacatttcgagggatacgcaagcaaaaaaaaaaaggaggtaaatagctctcacatggaacataggcgataataagtgcgactaggattgcatcagcaagaattagcgcgaatgcgaatggaactgcaggttttttgaatagatcggatcgattcgtctccttccccagccgacggctacgagaagctctcaaactcgccggtgatgaggcgcccgccatgaaaacagagcaaatcgcatcagcgtctagccaacgccgcgtaacagacaactacttccatattactactcttctaattagcccaaattaaatgagcctattgggcttcttgtcttagtcggtgtagagcccaattgttgttttattttttaataatgcaaaagtattaagcgataaataaataagcatcgcaatcgtcccaaaactgtgtgtatgcatcagacatgagcatatagagtaagcacgtgtccacactttttcacaaagttatctaaaaacaaaaaacaattaattagcattcgacgtgtacatatcactcgccacgtgtacaagagccttggcctttttgcttcttcttcttgtctattaatatcatctcctgattatactctcttttgaccaagctgcttcttctccatctatccaacatctcaaatctcagtaatcttgttccttcgattctgttttggacgtttgta

SEQ ID NO: 14 - DNA, 2000 bp, Arabidopsis thaliana
ttcggagaaaatgcagctaacgatatcaggaaaagtgattggtattgtgggagtgacagtagaagatgaagaggaagcaatactggggaagccaagatcccatcaccgttgagctctctctctctctctctcgtctccgtcgtgtggatctcgatctcgatctctctttgagccgcaacaactgatgagttgtctcagtctctctctctttctctctcctcttccttttcctctctctctttctctctctaaaagggctaagttttttaaaaaaatgtctaccaaactgatacaaagaggaagaagaagaaacactctgtacttttttttttaacaagagaagaaatgaaaacaaagatgtgaatttaagaaaaaaaataatgtgtcgacattacaaaactgtatttaatcatcatttgaaaacatttaatcatttgataaactatgtaacattacccccttaatcttctttccacctaacctttgtatcatttgttggtctttctattttataacattccttaaattattgaattaaaactatctaaattctgttaattactcacttgtaatccattttgttcactggaaatgtaaattttactgtcttattttgcaaaacactcttgaatttttttttattttttttctggtcaataagacacaaacatgttggattataaactattgtgatcataactctgttcggtaatgttttcaatatcaaatttgatagttggtgattggatttgtcaacatctctttttttttttgtttaatcctttgctaagttaagtcggccacatatatattgggtacgattttcataggtctttcctcacgccagaagtgttgttttattttgttgattgagttattaattattggaagcttttctttcaagcaaagtaaaatgcgtaataatgattagtcacatccaatggttagtcagtctattacaccgttaatcaagctctggtcatataatttttttatttttggaactaacacttattagtttaggtttccatcacctatttaattcgtaattcttatacatgcatataatagagatacatatatacaaatttatgatcatttttgcacaacatgtgatctcattcattagtatgcattatgcgaaaacctcgacgcgcaaaagacacgtaatagctaataatgttactcatttataatgattgaagcaagacgaaaacaacaacatatatatcaaattgtaaactagatatttcttaaaagtgaaaaaaaacaaagaaatataaaggacaattttgagtcagtctcttaatattaaaacatatatacataaataagcacaaacgtggttacctgtcttcatgcaatgtggactttagtttatctaatcaaaatcaaaataaaaggtgtaatagttctcgtcatttttcaaattttaaaaatcagaaccaagtgatttttgtttgagtattgatccattgtttaaacaatttaacacagtatatacgtctcttgagatgttgacatgatgataaaatacgagatcgtctcttggttttcgaattttgaactttaatagtttttttttttagggaaactttaatagttgtttatcataagattagtcacctaatggttacgttgcagtaccgaaccaattttttacccttttttctaaatgtggtcgtggcataatttccaaaagagatccaaaacccggtttgctcaactgataagccggtcggttctggtttgaaaaacaagaaataatctgaaagtgtgaaacagcaacgtgtctcggtgtttcatgagccacctgccacctcattcacgtcggtcattttgtcgtttcacggttcacgctctagacacgtgctctgtccccaccatgactttcgctgccgactcgcttcgctttgcaaactcaaacatgtgtgtatatgtaagtttcatcctaataagcatctcttaccacattaatttaaaacaaagaaaacatcaaa

SEQ ID NO: 15 - DNA, 2000 bp, Arabidopsis thaliana
atgcccgttacttgatcccacagggttataattcactgaaggattttttcccaaccatgaaaatgagaccaaatccacaatgctcgaatgtagcttgtctagagcggcaggtaacctgattaaatgcaaccacaaaactatctcagcttgtctttgttttgcgccttttgtttccatccatgttctggtgaaaaagttgaaatcccaaaatgcagaaagaatatatgcttgcaaaaccagaaagggatgctgctgctaaagctaagatggaagctgatgcatccacaactatcgatgaaggtccactccatgatgataatgagtggaacataaggtaataactcagtgaattagttaaaaatcaaaatttgtatttttgttatcatattttctcctttccagccatatttgcaacaagaaatctgatgtgtatctcatgtctttttatcagtgttgtcgatgatgaaaatgagaaggatactacaaaagctgcctcaagttcaggttacttaaacttgggactctatgtgctatccatgtgaaaatgtatcaaatataagtcctgttcttctgactgttatctaaattgatcctgtgttatgtgtagatacattacccgaagggcttactcgggagcttccagttgcagatgagtatgagaaagcaatagccatagcctcagggtcaggagaaacagaggaggaagatgaccttgaagatctgaagaaacagctcgaagctctcaacgctgcttgagtctttatgagggagaaaaaatagttgttttagcatatttatacatgaaagaggctacaagtgcaattgtgtatgcaaaaagagtgtgttaggattggtaaattggatctcatggacatataaacgtttttgatgatgacttctcccgtttttttagttgttaccaaagacaaaaagatacaattttgaagatgtttcatggatttattgtcataatcatccgaactagaacttagaaattcaaattagaaaaagattttgtccaatgatgatgatccaagtttggtttgtaatgtgtaaaccagcttttttggaggtaagatattaattgcgcggggaaatcaactagatgacgtgtgaatcgcgaattacaattacgtttttggatggagaactgtcttttttttcctttttatatccctaaaatacttttttgtacattcatttatatcaggaaaaacagagttgtaaccaaattattacagagatattcaaagatatttcaacctatcaactaaagatgacgtgtatattgagaatggcaattgcgtttatgagtgggaaaccaaattatttcattaacatgatctttaggaataccgctaataagagattactaatcactatttgaggttttaatttaaatctttgtagtcctgattttgaatatttcgttttagctaaatcagctccaatttttcccagttatcgtttgtcgaattcatattactgttaatttagttaacttccgaagactattagtacatgattcttcaacaaagcaaataaagaaaagaatcttgatgtcgttctctcaacaattcaacaaaacttggtctcgacgactcgaccgtgtgagatgtttgagaagtcagaacaccacacgtgtcgacataaagcccgcaccacgcacgcttttcgtctggatatatccaaattactttacagaaatcatttgtattgtttgttaataaaaactttgaaccactttgattactatattatttttttggaatttattctggtgacattaatgttctatcctccacgcggttctcataaaccgacttggcttctttatttttcctttatctttctctatatatttacacacacacgcatttgtgcagagatattcaaagattagaaacattcttgatagatacaaaaaacatttttcagacacaaaatcataaaatctttgcctttagtagatcaaagttctttacattaatcgttagaag

SEQ ID NO: 16 - DNA, 2000 bp, Arabidopsis thaliana
ttagctggggcaggtcggcttgatttcattgaggatctgcttgagcatcagaagacacttccacaaggtaggcgtgagggtttcattgttaggattataatgttgtatggcaaggctggaatgacgaagcaagcacttgatactttcttcaatatggatttatacggttgcaaaagatctgttaagtccttcaatgccgctcttcaagtcttatctttcaatcctgatctccacaccatctgggagttccttcacgacgccccatcgaagtatggcattgatattgatgcggtttcttttaacattgccattaaatctttctgtgagctgggtattcttgacggggcctatatggcaatgagggagatggagaaatcagggttaacaccagatgtagttacatatactacacttatatcggcgttatacaagcacgagaggtgtgtgattggaaatggattgtggaaccttatggtcctcaagggctgtaagccgaacctcaccacttttaatgttaggattcagtttttagtgaataggagacgagcttgggatgcaaatgatctgttgctgttgatgcctaaactccaggtggaaccggacagcataacatataacatggttatcaaagggtttttccttgcgaggttccctgacatggcggaaagagtttatacagcgatgcatggtaaggggtacaaacccaatttgaagatataccagacgatgattcactatttgtgcaaggcaggaaattttgatttggcttatacaatgtgcaaggattgtatgagaaagaagtggtacccaaacttggacacagttgagatgttgctcaaagggcttgtgaaaaaggggcagcttgatcaggctaagtcgatcatggagttagttcacagaagagtacctcctttcaggtcaaaacagttgctctcccttaagtccattttgtaatgtttacatgaaatggaacaaatttgctacatgtacacgacaatgcggccaaaagaattctttctttctttctcctgagtttattttgtatagctgtggtggaactggaaatgggatgtagcctcatattaggaggcacacttatcttccaatcagattcaactatttgttgttcaatgtgaataaagcttaggatgatttggaaacttcaatggagggatacccagtaagtcatggatgcctctacaatcttaagttgtgatagcacagaactagtttaccttttgtaatgctacttacaaaacatgttgttgatttcagctggagagcaaaatggcatacacaaagcagggaccatgtgctgaagaaaccaaaaggatcttcgtctgacgatgcaaaagctcttgagcacctggaaaaatgagttatgttgtaagcttcatctagccaaattaaaagcactaaacatataacaaaggatatcaacgttgatagctttttcttgatatagcacgaatgtaaacaacagagaatagaacagagacaacattaccagcaatgaatatttttattattatgtcgaataaatacattttcccacaagtgacagagcaaacaaaattcacatatttttgtctattacactttggtgtcagactgacttgttacgtagatttatggcggtggattataacattgtaactttaaatatcttttaacgtaagaaacaaaactccaaaattggtgtttatttttaaaaataaaaatttgagggggcatgtatgtttgtttagttgattcacgtgttctttgtgtctgaaaattctgaatttgatgattttgaaaaaggaaaaaaagatacgaggtatcctcaattccaaaaggatgaatgatacaactcatatatacaagattatagtaaaaagaagggatgcatataaagaaacatagacatcatcgccatccatcctcagatcagttccgtagtttactggatcacgacacacacacatatacacgtgtacatataagccattcttatcaaa

SEQ ID NO: 17 - DNA, 2000 bp, Arabidopsis thaliana
cacatttgacggcgaatcaagcgattctagaagcgacggagaagtcgaacaagattcacatcgtcgattttggaatcgttcaaggtatacaatggcctgctcttcttcaagctctagctactcgtacttctggtaaacccactcaaatccgggtctcgggtatacccgctccatctctcggtgaatctccggaaccgtcgttaatcgccaccggaaaccgcctccgtgatttcgccaaggttctggatctgaatttcgatttcatcccaattctcactcccatacatttacttaacgggtcaagtttccgggtcgacccggatgaagtactggccgtgaatttcatgctccagctctacaaattactcgacgagacgccgacgatagttgacaccgcactacggctcgccaaatcgttgaacccgagggtcgtcactctcggagaatacgaagtgagcttaaaccgggtcggtttcgctaaccgggtaaagaacgcgcttcaattctattccgcggttttcgaatcccttgaaccgaacttggggcgtgattcggaggagagagtgagagttgagcgagagttgttcggccggagaatctcgggtttgattggaccggagaaaaccggaattcatagagaaagaatggaagagaaagagcaatggcgggtattaatggagaatgccggttttgaatcggttaagctgagtaattacgcagtgagccaagcgaagattctattgtggaattacaattacagcaatttgtattcaattgttgaatctaagcctggcttcatctctttggcctggaacgatttacctctcctcactctttcttcctggcgataaccaaaccaaaccgatccggtattcttagttttgttttgttttcaatgttatttttggttagacaaatattcaattgttaatatactccgtggtcagagtgttttgtttttcttttagttcgaacgttgaattaattcaggggtaggttttgaattctctgaaccttatgtgttttttggtaacatcatttggatttgtgaactaggtttaaaaactggtcttagtcttgttgttttctcattagataatttaaactggtttgcttctttatttttggttggataaagtgaccggttctggtcatctgtttgagatgtaattactatttcataaaattaggaagttgaaagccaaatatatttgtaactactcttttatttgtaattttgctcaaaaaagtgatgaaatgtagttttgatatatgaatatctaccattatacataagtatatctgaacatggtacaacttatgaaagctaaatgtcaatacttgcaaagatataacaaatacaagttacatgaataagagatgtgtgttgaatttataagtgtcattttcttttcactttaaaacaaacttcatcttcttttgtttcttatgtgtcaaagttgccacagttgctctatttgagtctttcagtgtcagtctcagtcactgtactgattttacttttttttgttgagtgtgccaatgatgacatcactcccacgtcctccatccgtcttcttttaacggtcacgtggctcccaccctcttttctcgatgtctttaccgacttgttctagcccaacttacttgggccatttagattttttggtggcccaagttgctaaaagaggatttatcatagaaatctgaacccgttgcagcgctcaacacatgtcacagtcctgacaaacacgtattcaaatccttgttaagtcccgccacctgtcaccagagcaccacgaggcaaactctgatcaggacaccgtcgtactattatgtcggaagacaggaaagcttaattaagcttaaacctgacgtatttaacttcgttaactctaccttactaaagggttttaatttaaaacttatcatctcctcgtaagaataaaaactacttactctataaatttaagcttcaagaaacctccaaaagcagagaa

SEQ ID NO: 18 - DNA, 2000 bp, Arabidopsis thaliana
gggggttccacaatgcattgattgagtttgtgctgatgcagttacaacttgcttccgtctttttcacgtttcagctcgggacaaaaacacattactacggaaggacattgtttcatggaggcgcagagtatagaggaactggtcgagggtttgtggtcttccacgcgaaatttgctgagaactataggttctattcccgtagtcactttgtgaagggcattgaacttatgattcttctactggtatatcagatctttggtcaatcatataggggtgtggttacttatattctgatcactgtttcgatatggttcatggtggtgacatggctctttgctcctttccttttcaatccgtctggttttgagtggcaaaagattgttgatgactggacagactggaacaaatggatttataaccgaggaggaattggtgtccctcctgagaagagttgggaatcttggtgggagaaagagttagaacatcttagacattcaggggttcgtggtatcacccttgagatctttttggccttgcgcttcttcatctttcaatatggacttgtgtatcatctcagtaccttcaaaggaaaaaatcagagcttttgggtaagctaaccttataactacttccctgtgaatggtgaaactcttgattattgaacgataatgactctgttttcaggtttatggagcgtcatggtttgtgatcttattcattctactgattgtgaaggtatgaatctttaagcacattcacatttcaatttcttgccaaatcttaacattgtgttgctttcttctatgtaacgatcattgcagggtttgggcgttggaaggcgaagattcagcacgaatttccagcttctattcagaatcataaagggtcttgtgttcctcacatttgtggcaattctcataactttcctcgcacttcctctgataacaatcaaagacttattcatctgcatgcttgccttcatgccaactggatggggaatgcttctcgtaagtctttctcctccatcaaactcctctatttgtatctaagataccagttttgtctggtttttgtgacagttgctttgttttgtcctccacgtcagattgcacaagcgtgtaagcctctgatccaacagctagggatatggtcatcagttaggacgctagctcgaggctacgagatcgtgatgggcttgcttctgttcacaccagttgcgtttttggcctggtttccttttgtgtctgagttccagacaagaatgctctttaaccaagcctttagtagaggtctacaaatctctcgtattcttggtggtcaaagaaaagaccggtcttcaaaaaacaaggagtgacatgtacacatgtaaatggcgtatgtgtagatttaatggttatctgttattgtttccttcccaagtaatctatagttagaaagttcctgtaactttcatagatatatgttaattattttccatttccataagttcgcgtccattataatagaaagtgcatgagacgtatctttttcatatggaagacgaaaacattggttctccattctaaaagtcaaaaagaaaatgtatatcacttgaacttttccacataggtcatgataaagacaggactaatcaatcattcattgttgataatggtctcaattacctgttgctagttatttctacatttgtatagtgcaatactgtttttttttcctcttccaaataaaaactacagtactaaaagaaagttggtaatcataaaagttataaaactcaaataaaagcaattactctgtttctttttcactcttattttatatgactgaaatgaaattgtctatatgttgtttttgtttgtatgttcattgatatgtaaacatcaaatctgcaaaaaacaaccgcaagtttggatttatacgatgaagcgtttatctagtgagcgtcatcgcagacgtcacaagttatgtggtacaattttgcgtatgtttgatgaatacgc

SEQ ID NO: 19 - DNA, 23 bp, Artificial Sequence
  Description of Artificial Sequence synthetic DNA
cgccagggttttcccagtcacga

SEQ ID NO: 20 - DNA, 24 bp, Artificial Sequence
  Description of Artificial Sequence synthetic DNA
agcggataacaatttcacacagga